(19) United States
(12) Patent Application Publication
(10) Pub. No.: US 2025/0221783 A1
(43) Pub. Date: Jul. 10, 2025

(54) MEDICAL INSTRUMENT WRIST WITH MEASUREMENT OF GRIP POSITION

(71) Applicant: Intuitive Surgical Operations, Inc., Sunnyvale, CA (US)

(72) Inventors: Thad LIEB, Santa Barbara, CA (US); Matthew D. ROHR DANIEL, San Francisco, CA (US)

(73) Assignee: Intuitive Surgical Operations, Inc., Sunnyvale, CA (US)

(21) Appl. No.: 19/011,119

(22) Filed: Jan. 6, 2025

Related U.S. Application Data
(60) Provisional application No. 63/618,704, filed on Jan. 8, 2024.

(51) Int. Cl. A61B 34/35; A61B 34/20; A61B 34/30; A61B 90/00

(52) U.S. Cl. CPC A61B 34/35; A61B 2034/2061; A61B 2034/302; A61B 2034/305; A61B 2090/067

(57) ABSTRACT

A medical device includes a distal wrist link, and a first jaw and a second jaw each coupled to the distal wrist link. A shape fiber carrier is pivotably coupled to the distal wrist link and defines a shape fiber lumen. The medical device includes a shape fiber having a distal end fixedly coupled within the shape fiber lumen. The shape fiber carrier is indirectly coupled to the first jaw and the second jaw, and movement of the shape fiber carrier causes the shape fiber to produce a signal related to movement of at least one of the first jaw and the second jaw.

### CROSS-REFERENCE TO RELATED APPLICATIONS

This patent application claims priority to and the filing date benefit of U.S. Provisional Patent Application No. 63/618,704, filed Jan. 8, 2024, entitled “MEDICAL INSTRUMENT WRIST WITH MEASUREMENT OF GRIP POSITION,” which is incorporated by reference herein in its entirety.

### BACKGROUND

The embodiments described herein relate to medical devices, and more specifically to tools for minimally invasive surgery. More particularly, the embodiments described herein relate to medical devices that include wrist mechanisms and systems to measure grip position of the medical device.

Techniques for Minimally Invasive Surgery (“MIS”) employ instruments to manipulate tissue that can be either manually controlled or controlled via computer-assisted teleoperation. Some MIS instruments include a therapeutic or diagnostic end effector (e.g., forceps, a cutting tool, or a cauterizing tool) mounted on a wrist mechanism at the distal end of an instrument shaft. During an MIS procedure, the end effector, wrist mechanism, and the distal end of the shaft are inserted into a small incision or a natural orifice of a patient to position the end effector at a work site within the patient's body. The wrist mechanism can be used to change the end effector's orientation with reference to the shaft to perform the desired procedure at the work site. Wrist mechanisms generally provide specific mechanical degrees of freedom (“DOFs”) for movement of the end effector. For example, wrist mechanisms are able to change the pitch and yaw orientation of the end effector with reference to the shaft's longitudinal axis. A wrist may optionally provide a roll DOF for the end effector with reference to the shaft, or an end effector roll DOF may be implemented by rolling the shaft, wrist, and end effector together as a unit. An end effector may optionally have additional mechanical DOFs, such as grip or knife blade motion. In some instances, wrist and end effector mechanical DOFs may be combined to provide various end effector control DOFs. For example, U.S. Pat. No. 5,792,135 (filed May 16, 1997) discloses a mechanism in which wrist and end effector grip mechanical DOFs are combined to provide an end effector yaw control DOF.

To enable the desired movement of the distal wrist mechanism and end effector, instruments may include cables that extend through the shaft of the instrument and that connect the wrist mechanism to a mechanical structure configured to move the cables to operate the wrist mechanism and end effector. For teleoperated systems, the mechanical structure is typically motor driven and is operably coupled to a computer processing system to provide a user interface for a clinical user (e.g., a surgeon) to control the instrument as a whole, as well as the instrument's components and functions.

Further, the wrist mechanism generally provides specific degrees of freedom for movement of the end effector. For example, for forceps or other grasping tools, the wrist may be able to change the end effector pitch, yaw, and grip orientations with reference to the instrument shaft. More degrees of freedom could be implemented through the wrist but would require additional actuation members (e.g., cables) in the wrist and shaft, and these additional members compete for the limited space that exists given the size restrictions required by MIS applications. Components needed to actuate other degrees of freedom, such as end effector roll or insertion/withdrawal through movement of the main tube, also compete for space at or in the shaft of the device.

A conventional architecture for a wrist mechanism in a manipulator-driven medical device uses cables pulled in and payed out by a capstan in the proximal mechanical structure and thereby rotate the portion of the wrist mechanism that is connected to the capstan via the cables. For example, a wrist mechanism can be operably coupled to three capstans-one each for rotations about a pitch axis, a yaw axis, and a grip axis. Each capstan can be controlled by using two cables that are attached to the capstan so that one side pays out cable while the other side pulls in an equal length of cable. With this architecture, three degrees of freedom can require a total of six cables extending from the wrist mechanism proximally back along the length of the instrument's main shaft tube to the instrument's proximal mechanical structure. Efficient implementation of a wrist mechanism and proximal mechanical structure can be complicated because the cables must be carefully routed through the tool member, wrist mechanism, and proximal mechanical structure to maintain stability of the wrist throughout the range of motion of the wrist mechanism and to minimize the interactions (or coupling effects) of one rotation axis upon another. In addition, for MIS applications, it is desirable to have a compact instrument outer diameter to minimize the port size required for insertion of the instrument into a patient. However, with higher complexity mechanisms, it can be challenging to meet the desired instrument diameter (e.g., 12 mm, 8 mm, 5 mm, or smaller) while providing the required mechanical degrees of freedom.

Some wrist mechanisms include gripping tools or jaws and are part of a closed loop feedback system. In such a device the jaws can be used to grasp a tissue of a patient. It may be desirable to know the velocity and angular position of the jaws during use of the device. However, incorporating sensors into the jaws in order to obtain accurate velocity and angular position information for each of the jaws in a wristed medical device with a constrained device diameter (e.g., a diameter of approximately 5 mm or less) presents challenges. For example, to provide feedback regarding the velocity and angular position/orientation of each independently movable jaw may require each jaw to be directly coupled to a sensor. The inclusion of multiple sensors embedded in the jaws can require larger instrument size to accommodate routing of the sensors, which may be undesirable for a given port size, and also results in increased cost and complexity. If only one distally located sensor is available due to, for example, size constraints, or configuration of the jaws of medical device, it can be difficult to provide the sensor feedback needed to measure the position, velocity, and/or orientation of both of the jaws during use of the device.

Thus, a need exists for wrist mechanisms that provide improved sensor capabilities that can measure the position, velocity, and/or orientation of the jaws of the wrist mechanism while meeting design constraints of the medical device.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, a medical device includes a distal wrist link, and a first jaw and a second jaw each coupled to the distal wrist link. A shape fiber carrier is pivotably coupled to the distal wrist link and defines a shape fiber lumen. The medical device includes a shape fiber having a distal end fixedly coupled within the shape fiber lumen. The shape fiber carrier is indirectly coupled to the first jaw and the second jaw, and movement of the shape fiber carrier causes the shape fiber to produce a signal related to movement of at least one of the first jaw and the second jaw.

In some embodiments, the medical device includes an intermediate coupling mechanism coupled to the shape fiber carrier and the shape fiber carrier is indirectly coupled to the first jaw and the second jaw via the intermediate coupling mechanism. In some embodiments, the intermediate coupling mechanism is a differential coupled between at least one of the first jaw or the second jaw and the shape fiber carrier, and the shape fiber carrier is indirectly coupled to the first jaw and the second jaw via the differential. In some embodiments, the intermediate coupling mechanism includes a cam mechanism to indirectly couple the shape fiber carrier to the at least one of the first jaw or the second jaw. In some embodiments, the intermediate coupling mechanism includes a torsion spring to indirectly couple the shape fiber to the at least one of the first jaw or the second jaw.

In some embodiments, the signal produced by the shape fiber includes a signal associated with an average of a first yaw angle of the first jaw and a second yaw angle of the second jaw. In some embodiments, the shape fiber carrier is pivotably coupled to the distal wrist link about a first axis and the first jaw and the second jaw are each pivotably coupled to the distal wrist link about a second axis, where the second axis is offset from the first axis. In some embodiments, the distal end of the shape fiber is positioned between the first axis and the second axis. In some embodiments, the shape fiber carrier is indirectly coupled to the first jaw and the second jaw such that an angular position of the shape fiber carrier relative to the second axis is associated with an average yaw angle of the first jaw and the second jaw.

In some embodiments, the differential includes a housing, a first planet gear, a second planet gear, and a ring gear. The medical device further includes a gear train, a first sun gear and a second sun gear. The first sun gear is integral to the first jaw and is coupled to the housing. The second sun gear is integral to the second jaw and is coupled to the housing. The ring gear is integral to the housing and coupled to the gear train, and the gear train is coupled to the shape fiber carrier. The housing is configured to rotate when one of the first jaw or the second jaw rotates relative to the other such that a direction of the average of the positions of the jaws changes (e.g., with respect to the distal wrist link), and rotation of the housing causes rotation of the shape fiber carrier and the shape fiber. An orientation of the housing is associated with an average of a first yaw angle of the first jaw and a second yaw angle of the second jaw.

In some embodiments, the rotational movement of the shape fiber carrier is limited by the differential. In some embodiments, the rotational movement of the shape fiber carrier is limited to +/−45 degrees relative to a centerline of the shape fiber carrier. In some embodiments, the shape fiber carrier includes a gear operatively coupled to the differential.

In some embodiments, movement of the shape fiber carrier causes the shape fiber to produce a signal related to movement of the distal wrist link. In some embodiments, movement of the shape fiber carrier causes the shape fiber to produce a signal related to movement of a proximal articulating joint.

In some embodiments, a medical device includes a distal wrist link, and a first jaw and a second jaw each coupled to the distal wrist link. A position sensor is coupled to the distal wrist link and a differential is coupled to the position sensor, the first jaw, and the second jaw. Movement of one of the first jaw or the second jaw relative to the other produces a sensor movement of the position sensor via the differential, and the position sensor produces a signal related to the sensor movement.

In some embodiments, the signal produced by the position sensor is associated with an average of a first yaw angle of the first jaw and a second yaw angle of the second jaw. In some embodiments, the position sensor is a shape fiber, and the medical device includes a shape fiber carrier coupled to the differential. The shape fiber is at least partially disposed within a lumen of the shape fiber carrier.

In some embodiments, the differential includes a housing, a first planet gear, a second planet gear, and a ring gear. The medical device further includes a gear train, a first sun gear and a second sun gear. The first sun gear is integral to the first jaw and coupled to the housing, and the second sun gear is integral to the second jaw and coupled to the housing. The ring gear is integral to the housing and coupled to the gear train, and the gear train is coupled to the shape fiber carrier. The housing is configured to rotate when one of the first jaw or the second jaw rotates relative to the other such that a direction of the average of the positions of the jaws changes (e.g., with respect to the distal wrist link), and rotation of the housing causes rotation of the position sensor. An orientation of the housing is associated with an average of a first yaw angle of the first jaw and a second yaw angle of the second jaw.

In some embodiments, the position sensor produces a signal associated with the average of the first yaw angle of the first jaw and the second yaw angle of the second jaw based on the orientation of the housing. In some embodiments, rotational movement of the position sensor is limited by the differential. In some embodiments, rotational movement of the shape fiber carrier is limited by the differential. In some embodiments, the rotational movement of the shape fiber carrier is limited to +/−45 degrees relative to a centerline of the shape fiber carrier. In some embodiments, the position sensor is indirectly coupled to the first jaw and the second jaw such that an angular position of the position sensor is associated with an average of a first yaw angle of the first jaw and a second yaw angle of the second jaw.

In some embodiments, a medical device includes a distal wrist link, and a first jaw and a second jaw each coupled to the distal wrist link. A shape fiber carrier is coupled to the distal wrist link and defines a shape fiber lumen. A shape fiber extends from a position proximal to the distal wrist link to a distal end that is fixedly coupled within the shape fiber lumen. The shape fiber is spaced apart from each of the first jaw and the second jaw. Movement of the shape fiber carrier causes the shape fiber to produce a signal related to movement of at least one of the first jaw and the second jaw.

Other medical instruments, related components, medical device systems, and/or methods according to embodiments will be or become apparent to one with skill in the art upon review of the following drawings and detailed description. It is intended that all such additional medical devices, related components, medical device systems, and/or methods included within this description be within the scope of this disclosure.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of a user control console of the minimally invasive teleoperated surgery system shown in FIG. 1.

FIG. 3 is a perspective view of an optional auxiliary unit of the minimally invasive teleoperated surgery system shown in FIG. 1.

FIG. 4 is a perspective view of a manipulator unit, including a plurality of instruments, of the minimally invasive teleoperated surgery system shown in FIG. 1.

FIG. 5 is a diagrammatic top view of a distal end portion of a medical device according to an embodiment.

FIG. 6 is a diagrammatic top view of the distal end portion of a medical device according to another embodiment.

FIG. 7 is a side view of a medical device according to an embodiment, with a wrist assembly in a pitched configuration.

FIG. 8 is a top view of a distal end portion of the medical device of FIG. 7, with the wrist assembly in a straight configuration.

FIG. 9 is a perspective view of the distal end portion of the medical device of FIG. 7 shown with the wrist assembly in a pitched configuration (i.e., a distal link rotated relative to a proximal link of the medical device).

FIG. 10 a perspective view of the distal end portion of the medical device of FIG. 7 shown with a distal link rotated relative to a proximal link and with select components removed for illustration purposes.

FIG. 11 is a perspective view of a connector link of the medical device of FIG. 7.

FIG. 12 is a is a cross-sectional top perspective view of the distal end portion of the medical device of FIG. 7 with the distal link and proximal link in a straight configuration, with the jaws in a closed configuration, and with select components removed for illustration purposes.

FIG. 13 is a partial cross-sectional perspective view of the distal end portion of the medical device of FIG. 7 with the distal link rotated relative to the proximal link, with the jaws in the closed configuration, and with select components removed for illustration purposes.

FIG. 14 is a partial cross-sectional top perspective view of the distal end portion of the medical device of FIG. 7, with the distal link and proximal link in a straight configuration, the jaws in an open position, and with select components removed for illustration purposes.

FIG. 15 is a partial cross-sectional top perspective view of the distal end portion of the medical device of FIG. 7, showing another side of the medical device, with the distal link and proximal link in a straight configuration and with select components removed for illustration purposes.

FIG. 16 is a partial cross-sectional top view of the distal end portion of the medical device of FIG. 15 showing another side of the medical device, with the distal link and proximal link in a straight configuration and with select components removed for illustration purposes.

FIG. 17 is a perspective view of the medical device of FIG. 7 shown with the distal link rotated relative to the proximal link and select components removed for illustration purposes.

FIG. 18 is a perspective view of the medical device of FIG. 7 shown with the distal link rotated relative to the proximal link and select components removed for illustration purposes and showing an opposite side of the medical device than in FIG. 17.

FIG. 19 is a partial exploded perspective view of the medical device of FIG. 18.

FIG. 20 is a partial exploded perspective view of the medical device of FIG. 18 showing an opposite side of the medical device than in FIG. 19.

FIG. 21 is a further exploded view of the medical device of FIG. 18 showing the planet gears of the differential.

FIGS. 22A-22C are each a different perspective view of the differential of the medical device of FIG. 7.

FIG. 23 is a side view of a distal end portion of a medical device, according to another embodiment.

FIG. 24 is a perspective view of the distal end portion of the medical device of FIG. 23, with one of the tool members and the cables removed for illustration purposes.

FIG. 25 is a side view of a portion of the distal end portion of the medical device of FIG. 23 with select components removed for illustration purposes.

FIG. 26 is an exploded perspective view of a portion of the distal end portion of the medical device of FIG. 23.

FIG. 27 is an exploded perspective view of a portion of the distal end portion of the medical device of FIG. 23 showing an opposite side from what is shown in FIG. 26.

FIGS. 28A-28C each illustrate the distal end portion of the medical device of FIG. 23 with the tool members oriented in different positions relative to each other and/or to the distal link.

FIGS. 29A-29E are each a side view of the distal end portion of the medical device of FIG. 23 with a portion of one of the tool members removed for illustration purposes and each showing the tool members in a different position relative to each other and/or to the distal link.

FIG. 30 is a perspective view of a distal end portion of a medical device, according to another embodiment.

FIG. 31 is a perspective view of the distal end portion of the medical device of FIG. 30 with one of the tool members and the cables removed for illustration purposes.

FIG. 32 is a side view of a portion of the distal end portion of the medical device of FIG. 30 with select components removed for illustration purposes.

FIG. 33 is a side view of the portion of the distal end portion of the medical device of FIG. 32 with one of the torsion springs removed for illustration purposes and showing a portion of a shape fiber sensor.

FIG. 34 is an exploded perspective view of a portion of the distal end portion of the medical device of FIG. 30.

FIG. 35 is another exploded perspective view of a portion of the distal end portion of the medical device of FIG. 30.

FIG. 36A is a perspective view of the shape fiber carrier and intermediate coupling mechanism of the medical device of FIG. 30.

FIG. 36B is an exploded perspective view of the shape fiber carrier and intermediate coupling mechanism of FIG. 36A.

FIG. 37 is a side view of the distal end portion of the medical device of FIG. 30 with a portion of one of the tool members removed for illustration purposes and showing a shape fiber vector.

FIG. 38 is schematic illustration showing how a shape fiber carrier is balanced by an intermediate coupling mechanism that includes opposing springs.

FIG. 39 is a flowchart illustrating a method of assembling a medical device as having a shape fiber sensor.

FIG. 40 is a schematic illustration of a controller for use with a minimally invasive teleoperated surgery system according to an embodiment.

FIG. 41 is a flowchart illustrating a method of producing an adjusted output signal from a shape fiber sensor according to an embodiment.

FIG. 42 is a flowchart illustrating a method calibrating a shape fiber sensor of a medical instrument, according to an embodiment.

### DETAILED DESCRIPTION

The embodiments described herein can advantageously be used in a wide variety of grasping, cutting, and manipulating operations associated with minimally invasive surgery. In some embodiments, an end effector of a medical device (e.g., an instrument) can move with reference to a force transmission mechanism of the instrument in multiple mechanical DOFs. For example, the medical devices described herein can have one or more mechanical DOFs in the end effector itself, e.g., two jaws, each rotating with reference to a clevis (2 DOFs, which include a “grip” DOF when the jaws rotate in opposite directions towards each other in a closing manner) and a distal clevis that rotates with reference to a proximal clevis (one DOF).

In some embodiments, the medical devices of the present application enable distal end motion in three degrees of freedom (e.g., about a pitch axis, a yaw axis, and a grip axis) using only four tension elements (e.g., cables, bands, or the like), thereby reducing the total number tension elements required for articulation, reducing the space required within the shaft and wrist for routing tension elements, reducing overall cost, and enabling further miniaturization of the wrist and shaft assemblies to promote MIS procedures. As described herein, in some embodiments, a medical device includes a wrist assembly that includes a proximal link coupled to a distal link, which is coupled to an end effector. The distal link can rotate relative to the proximal link along a rolling arcuate contact surface of each of the distal and proximal links.

Medical devices described herein can include an end effector in the form of gripping tools or jaws at a distal end of the medical device that can be used to grasp tissue of a patient, grasp a needle (e.g., for suturing), or perform other operations. The end effector may be coupled to a wrist assembly and the wrist assembly may be operatively coupled to an instrument shaft that may be substantially rigid or may be a flexible member. In various embodiments, the medical device may have a maximum diameter of approximately 5 mm, and in other embodiments a maximum diameter between 4-6 mm, and in other embodiments a maximum diameter of approximately 8-12 mm.

The end effector and wrist assembly can be part of a closed loop feedback system for the medical device. Sensors embedded in the medical device can be integrated into a closed loop control system designed to reduce hysteresis of the medical device and improve the positional accuracy for articulation of the medical device. In various embodiments, the medical device can include one or more position or orientation sensors such as, for example, one or more shape fibers or shape fiber bundles (e.g., one or more optical fibers or fiber bundles having fiber Bragg gratings) to provide information associated with the position, orientation, and/or velocity of joints or links of the medical device (e.g., of the end effector, wrist joints, wrist links, and/or joints and links of the medical device shaft). Such sensors may also provide information regarding the position and/or orientation of the medical device relative to patient anatomy (e.g., position and/or orientation of the medical device relative to organs, cavities, and/or lumens through which the medical device may pass), as well as position and/or orientation information of the patient anatomy itself (e.g., position and/or orientation of organs, flexible lumens, movable soft tissue, etc.).

For position and/or orientation feedback of a minimally invasive medical device having an end effector with two jaws, it is desirable to have sensor information indicative of the position and/or orientation of both jaws. However, incorporating shape fibers into both jaws in a wristed medical device with a constrained device diameter (e.g., a diameter of approximately 5 mm or less) presents challenges. In some embodiments, the minimum allowable bend radius of a shape fiber in the medical device may be restricted to prevent breaking of the shape fiber during device articulation. For example, in some embodiments, the minimum allowable bend radius may be in the range of 4-5 mm. This can create challenges in routing a shape fiber through articulating jaws and an articulating wrist mechanism of a reduced diameter minimally invasive medical device that may move in pitch and/or yaw degrees of freedom. In addition, the shape fiber may be provided with a straight section at the distal end of the shape fiber to accurately transmit and reflect optical signals for position and orientation determination. For example, in some embodiments, the straight section may be at least 3 mm in length. This can create challenges in minimizing the length of the end effector and the minimum throw arm required for articulating the end effector.

The embodiments described herein overcome the challenges in providing end effector position, velocity, and/or orientation sensor feedback for minimally invasive medical devices with pitch and yaw articulation. Due to size constraints, routing limitations, and/or the configuration of the jaws and wrist of the medical device, the devices described herein may include a sensor/sensor bundle positioned at a distal end of the medical device wherein the sensor is spaced apart from the jaws of the medical device. For example, the sensor/sensor bundle may determine position, velocity, and/or orientation information of the jaws via indirect coupling to the jaws via one or more intervening elements (e.g., without the sensor/sensor bundle being embedded directly into the jaws). In some embodiments, only a single sensor/sensor bundle is positioned at a distal end of the medical device. The position sensor and mechanisms described herein can be used to sense the average position and average velocity of the two jaws during use of the medical device without being coupled directly to the jaws. In some embodiments, a spline gear differential is used to determine the average angular velocity and position of the jaws. In alternative embodiments, a cam actuated mechanism can be coupled to the jaws and used to determine the average angular velocity and position of the jaws.

More specifically, in some embodiments, a shape fiber carrier is pivotally coupled within a distal wrist link and is coupled to a spline gear differential positioned between the two jaws of the medical device. Each jaw has an integral sun gear that connects to one of two planet gears of the differential. The planet gears are located in a differential housing and are also geared to each other. The outside of the gear housing has an integral ring gear that connects to the distal shape fiber carrier via a gear train. The shape fiber carrier has a shape fiber lumen within which the shape fiber extends to a distal end of the lumen. Thus, the shape fiber carrier and shape fiber are not directly coupled to either of the jaws but rather are operatively coupled to the jaws via the differential.

When the average position of the jaws changes, the differential housing will be rotated accordingly, which in turn moves the shape fiber carrier and shape fiber disposed therein. In addition to providing this information about the position of the jaws, the shape fiber can also provide the orientation of the distal wrist link (e.g., relative to the proximal wrist link), as well as the position and velocity of the proximal wrist link, any elbow or other articulating joints and any flexible instrument parts that the shape fiber may pass through (e.g., a flexible instrument shaft). In this manner, the embodiments described herein allow for a single shape fiber sensor/sensor bundle to provide information about different aspects of the distal end of the medical device, including the jaw position. The embodiments described herein allow for efficient, compact end effector designs. In some embodiments, the tension elements that actuate the jaws are routed along an exterior portion of the jaws, and the differentials described may be packaged within the jaw bodies for a compact form. In some alternative embodiments, the gear train connecting the differential to the distal shape fiber carrier could be replaced with cables. In further alternative embodiments, in place of a differential, a cam actuated mechanism can be integrated into or coupled to the jaws and used to determine the average angular velocity and position of the jaws.

The measurement of jaw position is determined as the position sensor (e.g., shape fiber) bends in proportion to the average/differential between the two jaws. In the case of a shape fiber, the use of a differential limits the motion of the shape fiber to accommodate the limits on bend radius of the shape fiber and a straight section at a distal tip of the shape fiber. This also allows for the use of a single shape fiber/shape fiber bundle to determine the position of two jaws. The differential can alternatively be used with other types of position sensors.

As used herein, the term “distal” refers to direction towards a work site, and the term “proximal” refers to a direction away from the work site. Thus, for example, the end of a medical device that is closest to the target tissue would be the distal end of the medical device, and the end opposite the distal end would be the proximal end of the medical device.

Further, specific words chosen to describe one or more embodiments and optional elements or features are not intended to limit the disclosure. For example, spatially relative terms—such as “beneath”, “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, medical instrument, and variants thereof, can be interchangeably used.

Aspects of this disclosure are described with reference to a teleoperated surgical system. An example architecture of such a teleoperated surgical system is the da Vinci® surgical system, commercialized by Intuitive Surgical, Inc. of Sunnyvale, California. Examples of such surgical systems are the da Vinci Xi® surgical system (Model IS4000), da Vinci X® Surgical System (Model IS4200), and the da Vinci Si® surgical system (Model IS3000). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® surgical systems (e.g., the Model IS4000, the Model IS3000, the Model IS2000, the Model IS1200, the Model SP1099) are merely presented as examples, and they are not to be considered as limiting the scope of the inventive aspects disclosed herein. As applicable, inventive aspects may be embodied and implemented in both relatively smaller, hand-held, hand-operated devices that are not mechanically grounded in a world reference frame and relatively larger systems that have additional mechanical support that is grounded in a world reference frame.

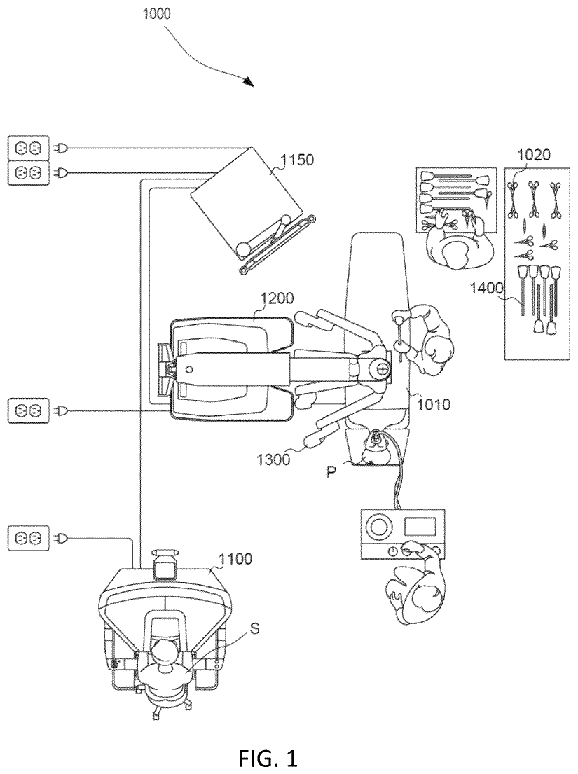

FIG. 1 is a plan view illustration of a teleoperated surgical system 1000 that operates with at least partial computer assistance (a “telesurgical system”). Both telesurgical system 1000 and its components are considered medical devices. Telesurgical system 1000 is a Minimally Invasive Robotic Surgical (“MIRS”) system used for performing a minimally invasive diagnostic or surgical procedure on a Patient P who is lying on an operating table 1010. The system can have any number of components, such as a user control unit 1100 for use by a surgeon or other skilled clinician S during the procedure. The MIRS system 1000 can further include a manipulator unit 1200 (popularly referred to as a surgical robot) and an optional auxiliary equipment unit 1150. The manipulator unit 1200 can include one or more arm assemblies 1300 that removably couple to surgical instruments 1400. The manipulator unit 1200 can manipulate at least one removably coupled instrument 1400 through a minimally invasive incision in the body or natural orifice of the patient P while the surgeon S views the surgical site and controls movement of the instrument 1400 through control unit 1100. An image of the surgical site is obtained by an endoscope (not shown), such as a monoscopic or stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces the instrument 1400 with another instrument 1400, for example from a tray 1020 in the operating room. Although shown as being used with the instruments 1400, any of the instruments described herein can be used with the MIRS 1000.

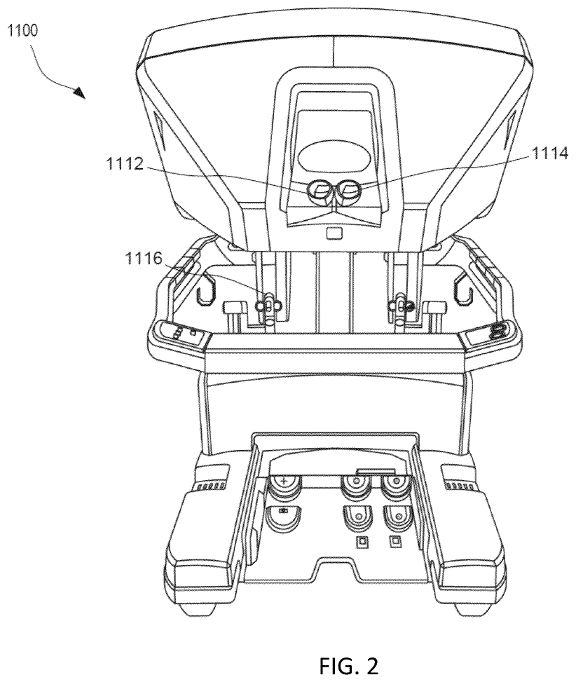

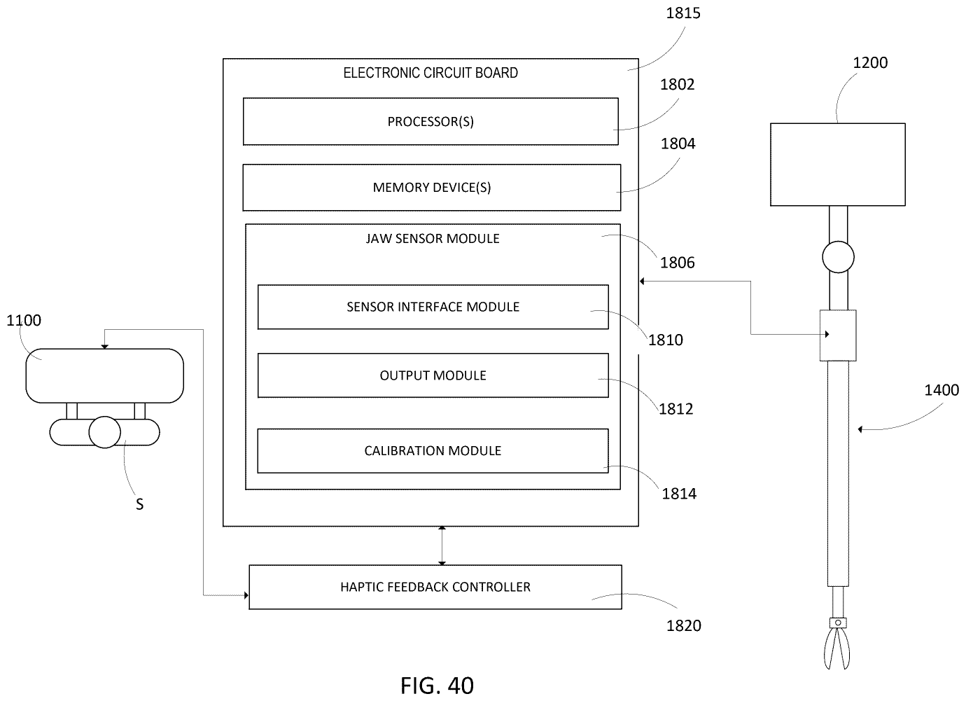

FIG. 2 is a perspective view of the control unit 1100. The user control unit 1100 includes a left eye display 1112 and a right eye display 1114 for presenting the surgeon S with a coordinated stereoscopic view of the surgical site that enables depth perception. The information presented to the surgeon S can be presented in the form of a graphical user interface through which inputs can be provided to a controller 1820 (FIG. 40). The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations may be provided from the instruments 1400 back to the surgeon's hand or hands through the one or more input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room from the patient, a completely different building from the patient, or other location remote from the patient, allowing for remote surgical procedures.

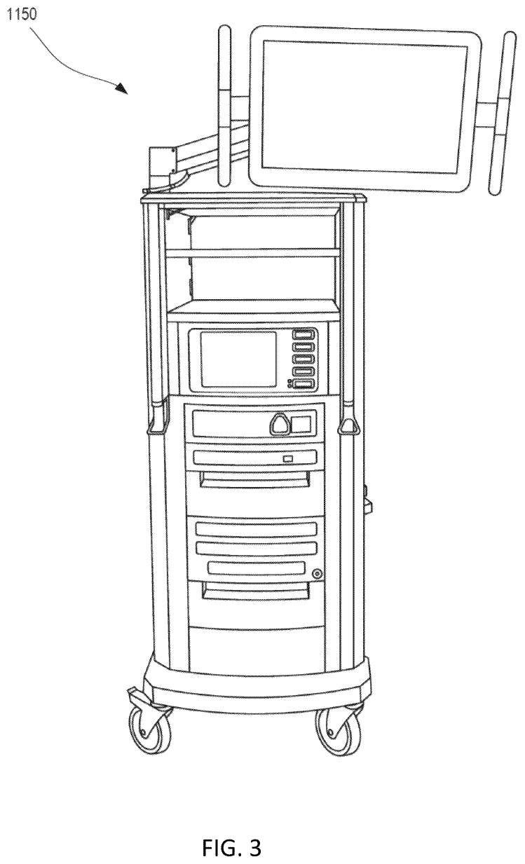

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

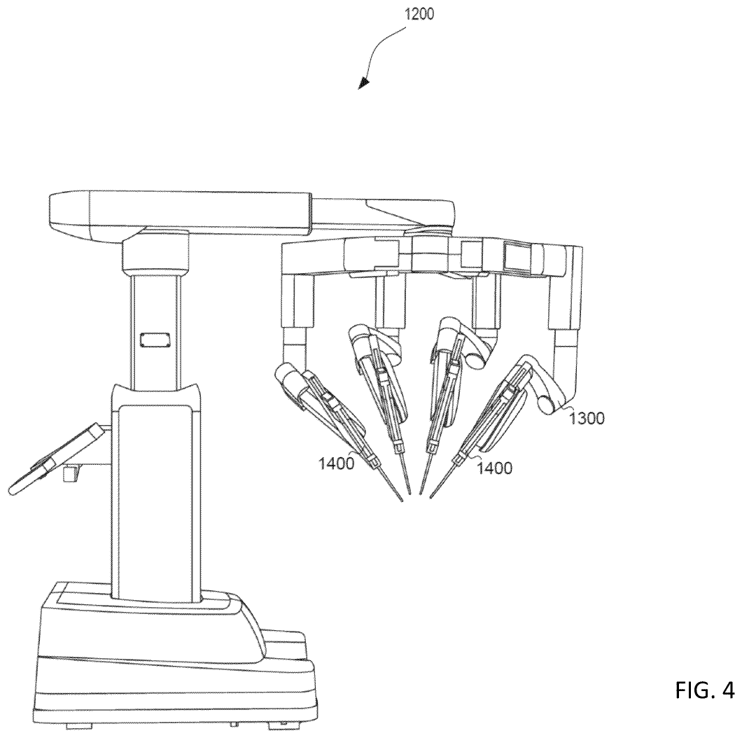

FIG. 4 shows a perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized. In various embodiments, the manipulator unit 1200 may be configured as a patient side cart with one or more arm assemblies 1300 supported and actuated by a single cart and/or individual manipulators supported and driven by separate carts. In other embodiments, the manipulator unit 1200 may be configured as a table mounted manipulator system wherein one or more arm assemblies 1300 are mounted to the operating table 1010 supporting the Patient P. In further embodiments, one or more arm assemblies 1300 may be coupled to the ceiling or other objects in the environment. It should be further appreciated that any of the arm assembly mounting configurations may be used in combination with each other.

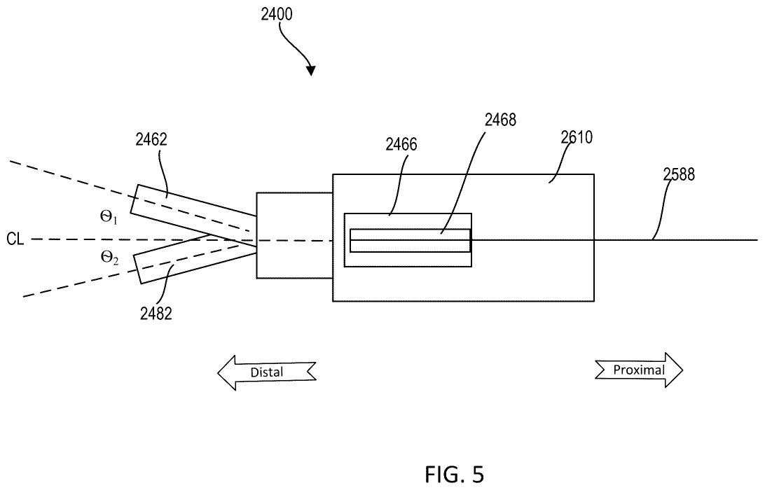

FIG. 5 is a schematic illustration of a portion of a medical device 2400 according to an embodiment. In some embodiments, the medical device 2400 or any of the components therein are optionally parts of an instrument of a surgical system that performs surgical procedures, and which surgical system can include a manipulator unit, a series of kinematic linkages, a series of cannulas, or the like. The medical device 2400 (and any of the instruments described herein) can be used in any suitable surgical system, such as the MIRS system 1000 shown and described above. The medical device 2400 includes a shaft (not shown), a proximal wrist link (not shown) coupled to the shaft, a distal wrist link 2610 coupled to the proximal wrist link, a first tool member 2462, a second tool member 2482, a shape fiber carrier 2466 that defines an interior lumen 2468, and a shape fiber 2588 extending distally within the shape fiber lumen 2468. The shape fiber 2588 can be a single optical fiber or a bundle of multiple optical fibers, which will be collectively referred to as a “shape fiber”. The shape fiber 2588 (and any of the shape fibers described herein) can be of the types shown and described in U.S. Pat. No. 8,358,883 B2 (filed Feb. 6, 2012), entitled “Fiber Optic Shape Sensor,” which is incorporated herein by reference in its entirety.

Although not shown in FIG. 5, the medical device 2400 can also include one or more tension elements (not shown) (which can be, for example, a cable, band or the like) coupled to the tool members 2462, 2482. The medical device 2400 is configured such that movement of the tension elements produces movement of the distal wrist link 2610, movement of the tool members 2462 or 2482, or both movement of the distal wrist link 2610 and movement of the tool members 2462 or 2482.

The tool members 2462, 2482 can rotate about a tool member rotation axis (not shown in FIG. 5). The tool members 2462, 2482 can be, for example, a pair of jaws, a cautery device, a cutting device, or other medical tool. The tension element(s) are coupled to the tool members 2462, 2482, extend from the tool members 2462, 2482, and are routed through the shaft and extend proximally to a force transmission mechanism (not shown in FIG. 5) of the medical device where they are coupled to drive components (not shown in FIG. 5) within the mechanical structure. The tension elements can be actuated by the drive components (not shown) such that tension on the tension elements urge one or both of the tool members 2462, 2482 to rotate about the tool member rotation axis as described in U.S. Provisional Patent Application Ser. No. 63/233,904, entitled “Surgical Instrument Cable Control and Routing Structures” incorporated herein by reference in its entirety.

For example, in some embodiments, the pitch of the tool members 2462, 2482 of the medical device 2400 can be changed by moving proximal ends of the tension elements to cause rotation of the distal wrist link 2610. Specifically, a force transmission mechanism (not shown, but which can be similar to the force transmission mechanism 4700 described further below) can pull in the proximal end of one of the tension elements while also releasing the same length of the proximal end of another of the tension elements to cause the rotation of the distal wrist link 2610. In other embodiments, the medical device 2400 can include additional tension element(s) to change the yaw and/or grip of the tool members 2462, 2482 relative to the distal wrist link 2610. For example, the yaw, grip, or both the yaw and grip can be changed by moving the proximal ends of the various tension elements. Thus, the medical device 2400 can include any suitable number of tension elements to produce the desired motion of the wrist link 2610 and tool members 2462, 2482. For example, in some embodiments, the medical device 2400 can include two tension elements to produce the desired pitch. In other embodiments, the medical device 2400 can include four tension elements and can operate in a manner similar to that shown and described in U.S. Patent Publication No. 2020/0390430, (filed Aug. 21, 2020), entitled “Low-Friction, Small Profile Medical Tools Having Easy-to-Assemble Components,” which is incorporated herein by reference in its entirety. In yet other embodiments, the medical device 2400 can include six tension elements and can operate in a manner similar to that shown and described in U.S. Patent Publication No. 2020/0390430 incorporated herein by reference.

The tool members 2462, 2482 can cooperatively perform gripping, shearing, or cutting functions. Thus, the tool member rotation axis can also function as a cutting axis as tool members rotate in opposition. Thus, in some embodiments, the medical device 2400, can provide at least three degrees of freedom (i.e., yaw motion about the tool member rotation axis, pitch rotation about the distal link rotation axis or proximal link rotation axis, and a cutting or gripping motion about the tool member rotation axis).

The shape fiber carrier 2466 is operatively coupled to the tool members 2462 and 2482 via an intermediate coupling mechanism, such as a differential, a cam mechanism, a torsion spring mechanism, or other suitable intervening structure (not shown). A distal end of the shape fiber 2588 is fixedly coupled to the shape fiber carrier 2466 within the shape fiber lumen 2468 at a distal end of the shape fiber lumen 2468. Thus, the shape fiber carrier 2466 and the shape fiber 2588 are indirectly coupled to the first and second tool members 2462, 2482. In other words, the shape fiber carrier 2466 and the shape fiber 2588 are spaced apart from each of the first tool member 2462 and the second tool member 2482. In operation, the intermediate coupling mechanism is coupled to the tool members 2462, 2482, and the intermediate coupling mechanism rotates as a result of rotation of one or both of the tool members 2462, 2482. The shape fiber carrier 2466 is coupled to the intermediate coupling mechanism. Rotation of the intermediate coupling mechanism causes the shape fiber carrier 2466 and the shape fiber 2588 coupled thereto to also bend proportionally. The bending of the shape fiber 2588 produces a sensor signal indicative of the amount of bending.

In some embodiments, the shape fiber 2588 has a straight section at a distal end of the shape fiber 2588 that facilitates accurate measurement of bends in the other portions of the shape fiber and can also minimize reflections of light (e.g., laser light). The straight section can have a predetermined length (e.g., of at least 3 mm, between 3 mm and 6 mm), and the shape fiber lumen 2468 is sized to accommodate the straight section. In some embodiments, the shape fiber 2588 can have a minimum bend radius of, for example, 5 mm to 6 mm. The shape fiber 2588 is coupled to the shape fiber carrier 2466 such that movement of the shape fiber carrier 2466 causes the shape fiber 2588 to bend and produce a signal related to movement of the first tool member 2462, the second tool member 2482, or both. More specifically, when the position or orientation of one or both of the tool members 2462, 2482 changes, the shape fiber carrier 2466 will be caused to move due to the shape fiber carrier 2466 being operative coupled to the tool members 2462, 2482. This will cause the shape fiber 2588, which is fixedly coupled to the shape fiber carrier 2466 to move and to produce a signal related to movement of at least one of the first tool member 2462 or the second tool member 2482.

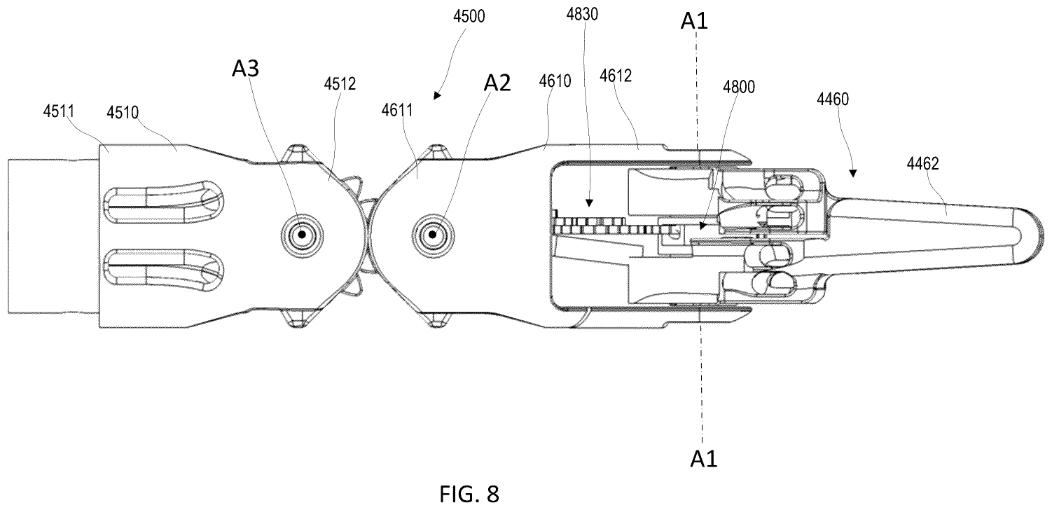

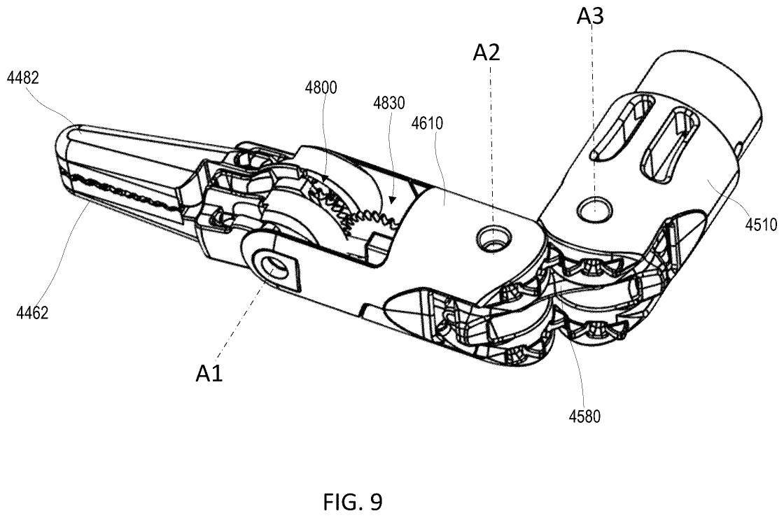

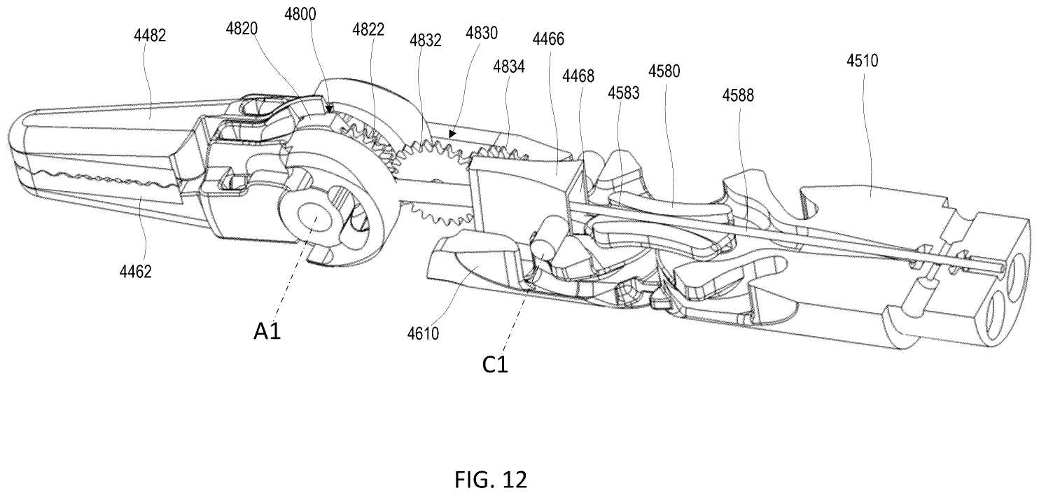

In some embodiments, the signal produced by the shape fiber 2588 includes a signal associated with an average of a first yaw angle θ₁ of the first tool member 2462 and a second yaw angle θ₂ of the second tool member 2482. As shown in FIG. 5, the yaw angle is a measure of the tool members with respect to a centerline CL of the distal wrist link 2610. In some embodiments, the shape fiber carrier 2466 is pivotably coupled to the distal wrist link 2610 about a first axis (see, e.g., carrier axis C1 in FIG. 12 discussed below), and the first tool member 2462 and the second tool member 2482 are each pivotably coupled to the distal wrist link 2610 about a second axis (see, e.g., axis A1 in FIGS. 8 and 9 discussed below), where the second axis is offset from the first axis. In this manner, the shape fiber carrier 2466 can move relative to the distal wrist link 2610 and relative to each of the first tool member 2462 and the second tool member 2482 during use. In some embodiments, the distal end of the shape fiber 2588 is positioned between the first axis and the second axis. In some embodiments, the shape fiber carrier 2466 is indirectly coupled to the first tool member 2462 and the second tool member 2482 such that an angular position of the shape fiber carrier 2466 relative to the second axis is associated with an average yaw angle of the first tool member 2462 and the second tool member 2482.

In some embodiments, an intermediate coupling mechanism such as a differential mechanism (not shown in FIG. 5) is coupled to the first tool member 2462 and the second tool member 2482, and the shape fiber carrier 2466 is pivotally coupled to the differential mechanism via a gear train (or other mechanical coupling). The differential mechanism can be, for example, operatively coupled between the first tool member 2462 and the second tool member 2482. Such an embodiment including a differential is described herein with reference to medical device 4400. The measurement of the position of the tool members 2462, 2482 (e.g., an angular position of the tool members 2462, 2482 with respect to the shaft of the medical device 2400) is determined as the shape fiber 2588 bends in proportion to the average/differential between the two tool members 2462, 2482. In some embodiments, the use of a differential limits the motion of the shape fiber 2588 to accommodate the limits on the bend radius of the shape fiber 2588 and to accommodate the straight section at the distal tip of the shape fiber 2588. In some embodiments, the rotational movement of the shape fiber carrier 2466 is limited by the differential. In some embodiments, the rotational movement of the shape fiber carrier 2466 is limited to +/−45 degrees relative to a centerline (not shown in FIG. 5) of the shape fiber carrier 2466. In some embodiments, the shape fiber carrier 2466 includes a gear (not shown) operatively coupled to the differential. In some embodiments, movement of the shape fiber carrier causes the shape fiber to produce a signal related to movement of the distal wrist link. In some embodiments, movement of the shape fiber carrier causes the shape fiber to produce a signal related to movement of a proximal articulating joint.

The differential mechanism can also allow for the use of a single shape fiber 2588 to determine the position of the two tool members 2462 and 2482. Thus, as described herein, a single shape fiber 2588 can be used to determine the position and orientation of the tool members 2462, 2482 (e.g., with respect to the shaft of the medical device 2400), with the shape fiber 2588 not being directly coupled to the tool members 2462, 2482.

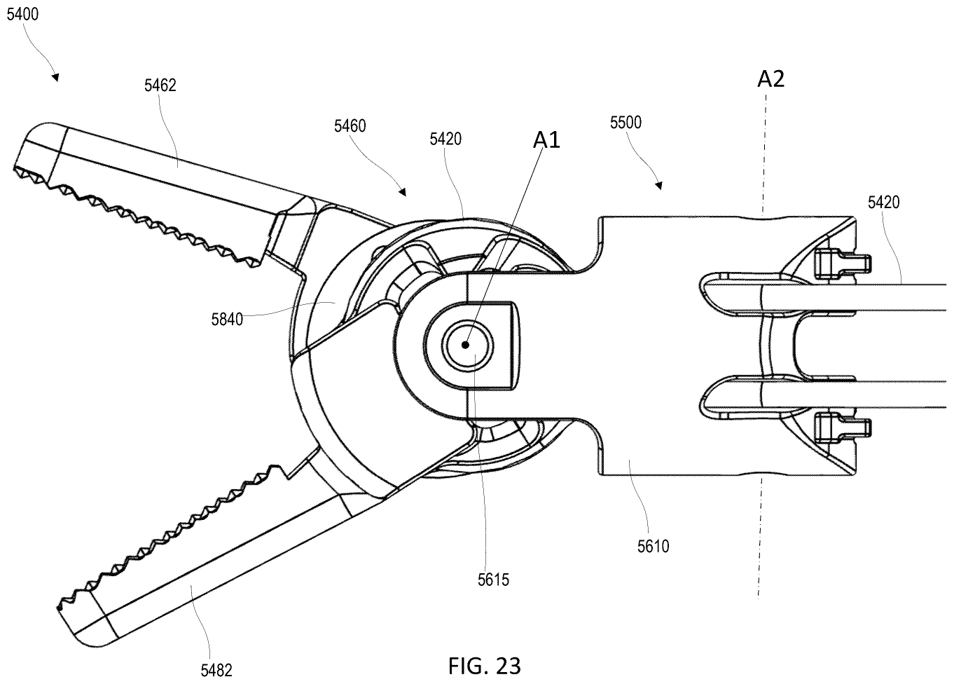

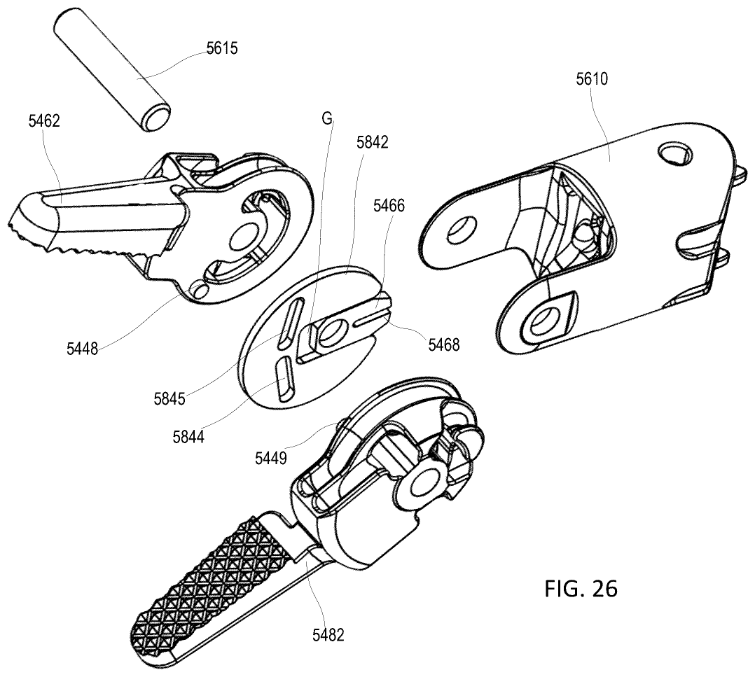

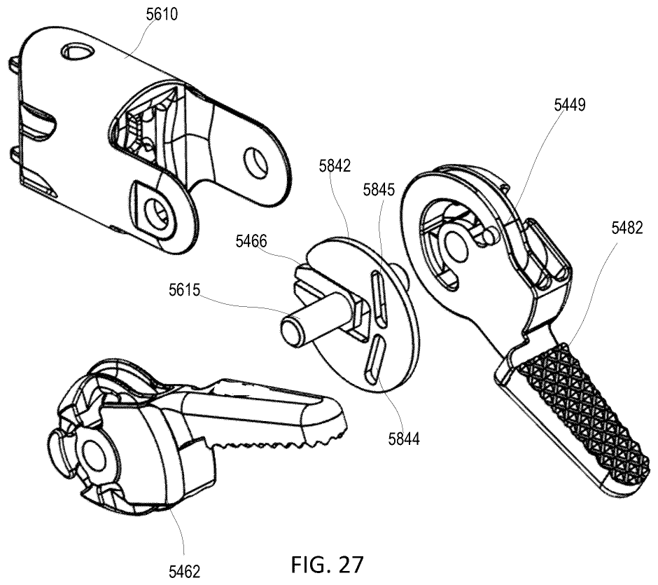

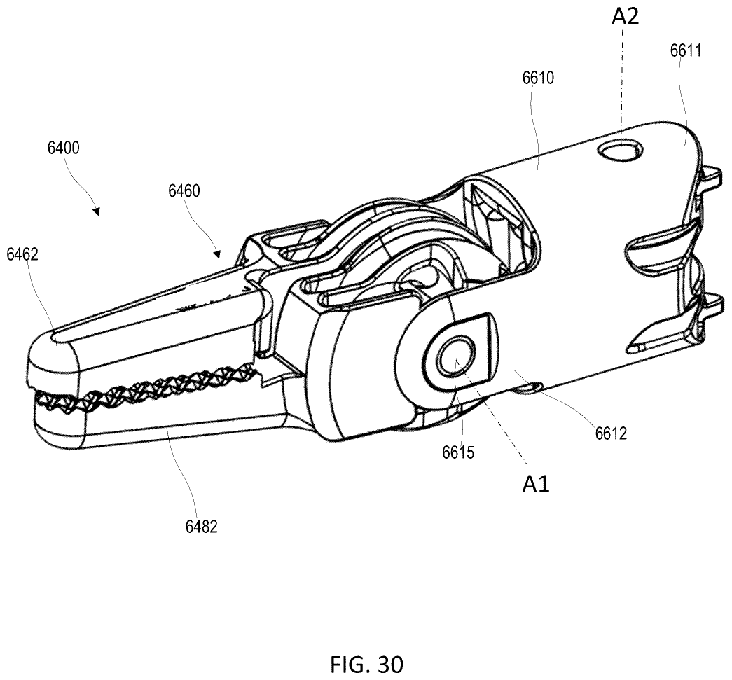

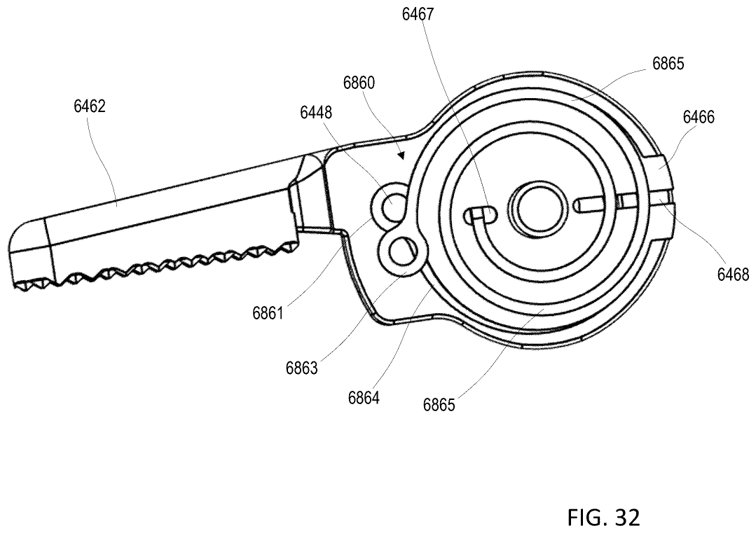

In alternative embodiments, in place of a differential, other intermediate coupling mechanisms can be coupled between the tool members 2462 and 2482 and the shape fiber carrier 2466. For example, a cam actuated mechanism can be coupled to the tool members and used to average the tool member's positions. Such an embodiment is shown in FIGS. 23-29E and described below. In another example, a torsion spring mechanism can be coupled to the tool members and used to average the tool member's positions as shown and described below with reference to FIGS. 30-38. In some alternative embodiments, the gear train connecting the differential to the distal shape fiber carrier could be replaced with cables. In some alternative embodiments, the differential can be used with other types of position sensors, such as for example, a hall effect sensor.

In some embodiments, the medical device 2400 can also include a connector link (not shown in FIG. 5) coupled between the distal wrist link 2610 and the proximal wrist link (not shown in FIG. 5). More specifically, the connector link includes a proximal end portion coupled to the proximal wrist link at the proximal link rotation axis, and a distal end portion coupled to the distal wrist link 2610 at the distal link rotation axis. Thus, the distal wrist link 2610 is rotatable with reference to the connector link about the distal link rotation axis, and the connector link is rotatable with reference to the proximal wrist link about the proximal link rotation axis. Through its coupling to the connector link, the distal wrist link 2610 is also rotatable about the proximal wrist link rotation axis. An embodiment including a connector link is described in more detail below with reference to medical device 4400.

In some embodiments, the connector link defines an internal pathway through which the shape fiber 2588 can extend. In some embodiments, other elongate elements can extend through the internal pathway of the connector link, such as, for example, an electrical wire, a cable, a tension element or the like. In some embodiments, the internal pathway includes a tapered entry at a proximal end of the internal pathway. In some embodiments, a distal end of the connector link is disposed distally of the distal link rotation axis.

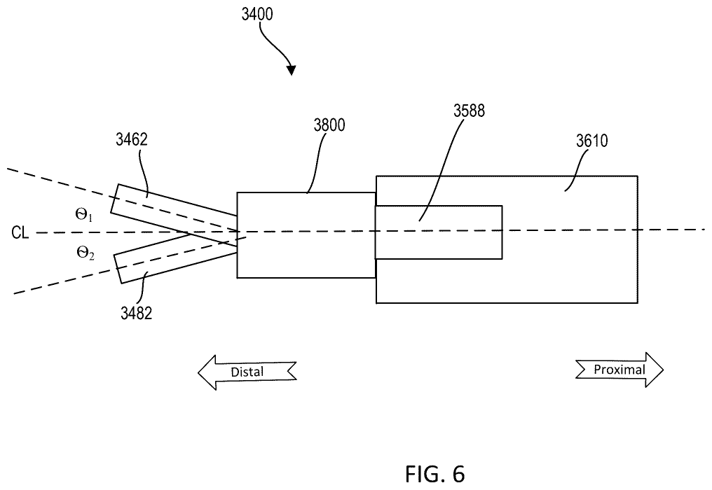

FIG. 6 is a schematic illustration of a portion of a medical device 3400 according to another embodiment. In some embodiments, the medical device 3400 or any of the components therein are optionally parts of an instrument of a surgical system that performs surgical procedures, and which surgical system can include a manipulator unit, a series of kinematic linkages, a series of cannulas, or the like. The medical device 3400 (and any of the instruments described herein) can be used in any suitable surgical system, such as the MIRS system 1000 shown and described above. The medical device 3400 can include a shaft (not shown in FIG. 6), a proximal wrist link (not shown) coupled to the shaft, a distal wrist link 3610 coupled to the proximal wrist link, a first tool member 3462, a second tool member 3482, a position sensor 3588, and a differential mechanism 3800 (also referred to herein as “differential”) coupled between the tool members 3462 and 3482 and the position sensor 3588. The position sensor 3588 is coupled to or at least partially within the distal wrist link 3610. The position sensor 3588 can be, for example, a shape fiber (such as the shape fiber 2488 described above), or a hall effect sensor.

Although not shown in FIG. 6, the medical device 3400 can also include one or more tension elements (not shown in FIG. 6) (which can be, for example, a cable, band or the like) coupled to the tool members 3462, 3482. The medical device 3400 is configured such that movement of the tension elements produces movement of the distal wrist link 3610, movement of the tool members 3462 or 3482, or both movement of the distal wrist link 3610 and movement of the tool members 3462 or 3482.

The tool members 3462, 3482 can rotate about a tool member rotation axis (not shown in FIG. 6). The tool members 3462, 3482 can be, for example, a pair of jaws, a cautery device, a cutting device, or other medical tool. The tension element(s) are coupled to the tool members 3462, 3482, extend from the tool members 3462, 3482 and are routed through the shaft and extend proximally to a mechanical structure (not shown in FIG. 6) of the medical device where they are coupled to drive components (not shown in FIG. 6) within the mechanical structure. The tension elements can be actuated by the drive component (not shown) such that tension on the tension elements one or both of the tool members 3462, 3482 to rotate about the tool member rotation axis.

The tool members 3462, 3482 can cooperatively perform gripping, shearing, or cutting functions. Thus, the tool member rotation axis can also function as a cutting axis as tool members rotate in opposition. Thus, in some embodiments, the medical device 3400, can provide at least three degrees of freedom (i.e., yaw motion about the tool member rotation axis, pitch rotation about the distal link rotation axis or proximal link rotation axis, and a cutting or gripping motion about the tool member rotation axis).

The differential 3800 is coupled to the first tool member 3462 and the second tool member 3482, and the position sensor 3588 is coupled to the differential 3800. The differential 3800 rotates responsive to rotation of the first tool member 3462 and/or the second tool member 3482. The position sensor 3588 bends responsive to the rotation of the differential 3800. As described herein, the differential 3800 allows for the first tool member 3462 and the second tool member 3482 to rotate independently of each other while the differential 3800 is coupled to (and produces movement in) the position sensor 3588. In some embodiments, the differential 3800 can be coupled to the position sensor 3588 via an optional gear train (not shown in FIG. 6) positioned in series between the tool members 3462, 3482 and the position sensor 3588. Thus, the position sensor 3588 is indirectly coupled to the tool members 3462 and 3482 via the differential 3800 and the optional gear train. Said another way, the position sensor 3588 is spaced apart from each of the first tool member 3462 and the second tool member 3482 but can be used to determine an orientation and position of the tool members 3462, 3482.

During use of the medical device 3400, movement of one of the first tool member 3462 or the second tool member 3482 relative to the other produces a movement of the position sensor 3588 via the differential 3800, and the position sensor 3588 produces a signal related to the movement of the position sensor 3588. In some embodiments, the signal produced by the position sensor 3588 is associated with an average of a first yaw angle θ₁ of the first tool member 3462 and a second yaw angle θ₂ of the second tool member 3482, as shown in FIG. 6. As described herein, the yaw angle is a measure of the tool members with respect to a centerline CL of the distal wrist link 3610.

In some embodiments, the differential 3800 is configured such that the ratio of the movement of the first tool member 3462 and the second tool member 3482 to the movement of the position sensor 3588 is greater than 1:1. In this manner, the differential 3800 can reduce the amount of movement transmitted to the position sensor 3588 thereby accommodating space constraints within the medical device 3400. In some embodiments the ratio of movement of the tool members 3462, 3482 to the position sensor 3588 is about 2:1. In other embodiments, the ratio of movement of the tool members 3462, 3482 to the position sensor 3588 is about 3:1. In some embodiments, rotational movement of the position sensor 3588 is limited by the differential 3800. In some embodiments, the rotational movement of the tool members 3462, 3482 may be each +/−110 degrees, while the rotational movement of the position sensor 3588 may be limited to +/−45 degrees relative to a centerline of the position sensor 3588 (or centerline CL of the distal wrist link 3610).

In some embodiments, the position sensor 3588 is a shape fiber, and the medical device includes a shape fiber carrier (not shown in FIG. 6) coupled to the differential 3800. The shape fiber can include a single shape fiber or a bundle of multiple shape fibers. The position sensor 3588 can be at least partially disposed within a lumen of the shape fiber carrier.

In some embodiments, the differential 3800 includes a housing, a first planet gear, a second planet gear, and a ring gear (each not shown in FIG. 6). The medical device further includes a gear train, a first sun gear and a second sun gear (each not shown in FIG. 6). The first sun gear is integral to the first tool member 3462 and is coupled to the first planet gear within the housing of the differential, and the second sun gear is integral to the second tool member 3482 and coupled to the second planet gear within the housing of the differential. The first and second planet gears are also geared to each other within the housing. The ring gear is integral to an exterior portion of the housing and is coupled to the gear train, which is indirectly coupled to the position sensor 3588. The housing is configured to rotate when one of the first tool member 3462 or the second tool member 3482 rotates relative to the other such that a direction of the average of the positions of the tool members 3462, 3482 changes (e.g., with respect to the distal wrist link), and rotation of the housing causes rotation of the position sensor 3588. An orientation of the housing is associated with an average of a first yaw angle of the first tool member 3462 and a second yaw angle of the second tool member 3482.

When the direction of the average of the positions of the tool members 3462, 3482 changes, the differential housing will be rotated accordingly, which in turn moves position sensor 3588 operatively coupled thereto. The position sensor 3588 can also provide the orientation of the distal wrist link 3610 (e.g., with respect to a proximal wrist link) and can also provide position and velocity data associated with the proximal wrist link, any elbow or other articulating joints and any flexible instrument parts that the position sensor may pass through (e.g., a flexible instrument shaft). In some embodiments, the tension elements that actuate the jaws run along an exterior portion the jaws, and the differential is packaged within the tool members 3462, 3482. In alternative embodiments, the gear train connecting the differential 3800 to the distal position sensor 3588 could be replaced with cables.

As described above, the differential 3800 can allow for the use of a single position sensor 3588 to determine the position of the two tool members 3462 and 3482. As described herein, a single position sensor 3588 can be used to determine the position and orientation of the tool members 3462, 3482, with the position sensor 3588 being indirectly coupled to the tool members 3462, 3482.

In some embodiments, the medical device 3400 can also include a connector link (not shown in FIG. 6) coupled between the distal wrist link 3610 and the proximal wrist link (not shown in FIG. 6). More specifically, the connector link includes a proximal end portion coupled to the proximal wrist link at the proximal link rotation axis, and a distal end portion coupled to the distal wrist link 3610 at the distal link rotation axis. Thus, the distal wrist link 3610 is rotatable with reference to the connector link about the distal link rotation axis, and the connector link is rotatable with reference to the proximal wrist link about the proximal link rotation axis. Through its coupling to the connector link, the distal wrist link 3610 is also rotatable about the proximal wrist link rotation axis. An embodiment including a connector link is described in more detail below with reference to medical device 4400.

In some embodiments, the connector link defines an internal pathway through which the position sensor 3588 can extend. In some embodiments, other elongate elements can extend through the internal pathway of the connector link, such as, for example, an electrical wire, a cable, a tension element or the like. In some embodiments, the internal pathway includes a tapered entry at a proximal end and a distal end of the internal pathway. In some embodiments, a distal end of the connector link is disposed distally of the distal link rotation axis.

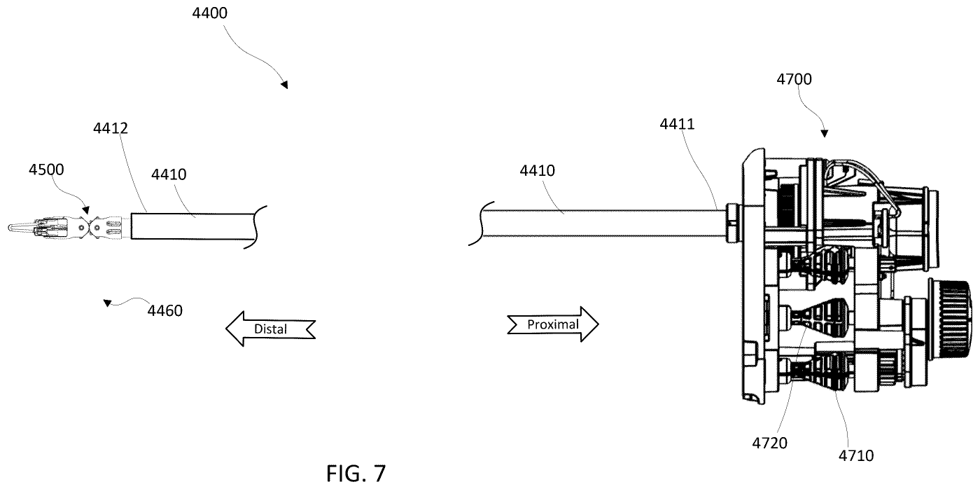

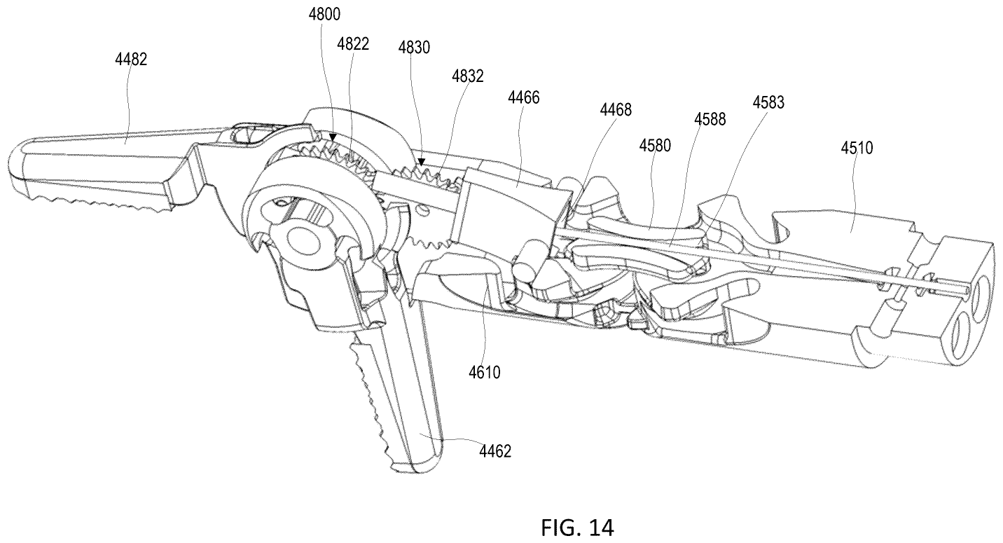

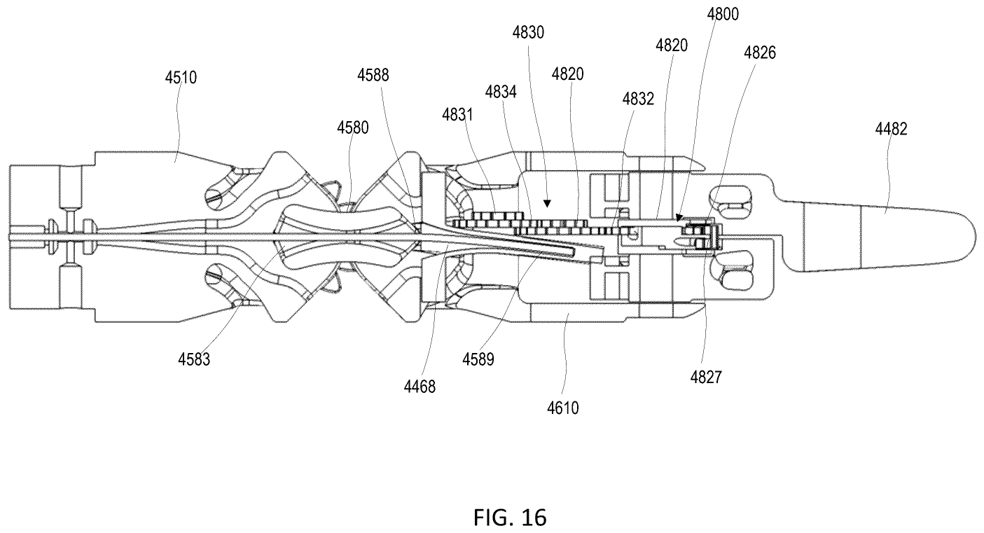

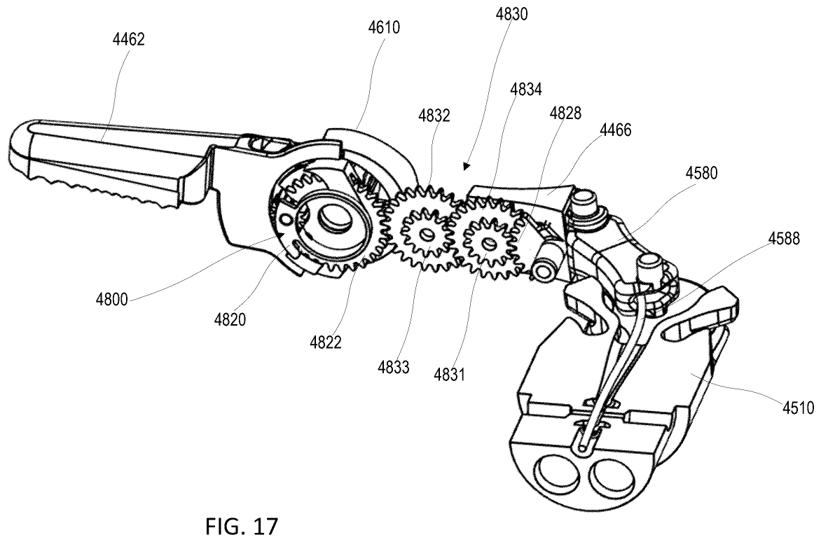

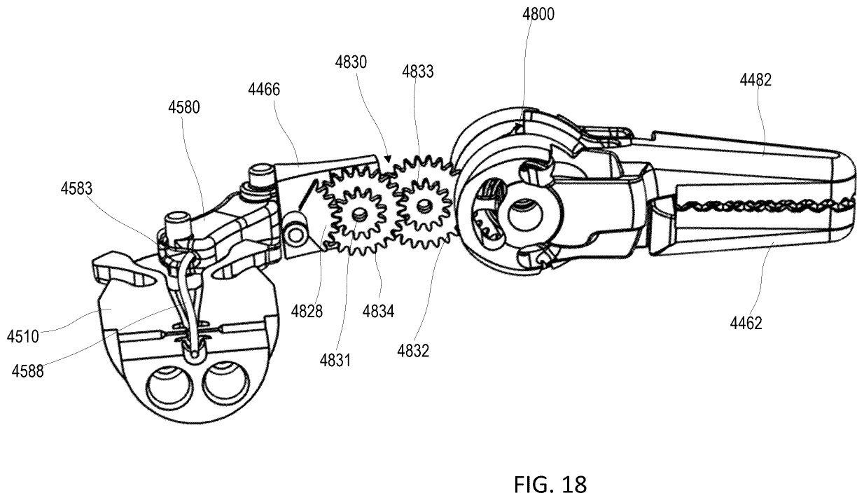

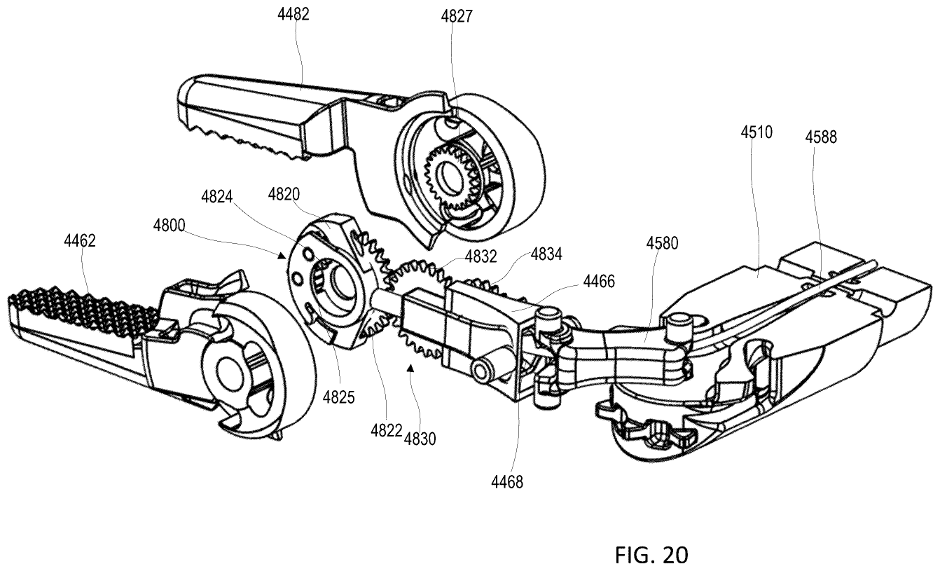

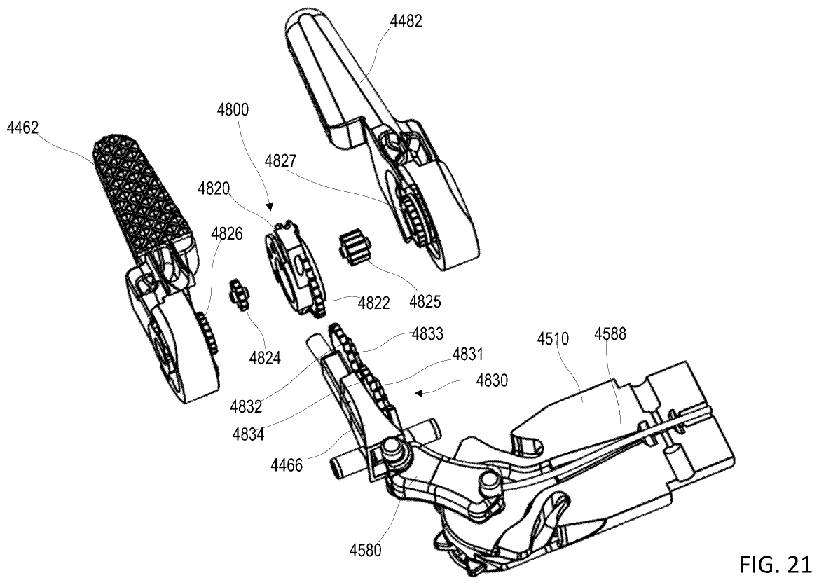

FIGS. 7-22C are various views of a medical device 4400 (and portions thereof), according to an embodiment. In some embodiments, the medical device 4400 or any of the components therein are optionally parts of an instrument for a surgical system that performs surgical procedures, and which surgical system can include a manipulator unit, a series of kinematic linkages, a series of cannulas, or the like. The medical device 4400 (and any of the instruments described herein) can be used in any suitable surgical system, such as the MIRS system 1000 shown and described above. As shown in FIG. 7, the medical device 4400 includes a force transmission mechanism 4700, a shaft 4410, a wrist assembly 4500, and an end effector 4460. The medical device 4400 can also include one or more tension elements (not shown in FIGS. 7-22C). The tension elements can be, for example, cables, bands or the like) that couple the force transmission mechanism 4700 to the wrist assembly 4500 and end effector 4460.

Referring to FIGS. 8 and 9, the medical device 4400 is configured such that movement of one or more of the tension elements produces rotation of the end effector 4460 about a first rotation axis A1 (also referred to herein as “tool member axis”), which functions as a yaw axis (the term yaw is arbitrary), rotation of the wrist assembly 4500 about a second rotation axis A2 (also referred to as “distal wrist rotation axis”) or a third rotation axis A3 (also referred to as “proximal wrist rotation axis”), a cutting (or gripping) rotation of the tool members of the end effector 4460 about the first rotation axis A1, or any combination of these movements. FIG. 8 illustrates the distal wrist link 4610 and the proximal wrist link 4510 in a straight configuration. FIG. 9 illustrates the distal wrist link 4610 rotated relative to the proximal wrist link 3510 (i.e., in a “pitched” configuration), with tool members 4462, 4482 in a closed configuration. Changing the pitch or yaw of the medical device 4400 can be performed by manipulating the tenson elements in a similar manner as that described with reference to the device 2400 described above and described in U.S. Provisional Patent Application Ser. No. 63/233,904, entitled “Surgical Instrument Cable Control and Routing Structures” and U.S. Patent Publication No. 2020/0390430, (filed Aug. 21, 2020), entitled “Low-Friction, Small Profile Medical Tools Having Easy-to-Assemble Components,” the disclosure of each of which is incorporated herein by reference above.

The force transmission mechanism 4700 produces movement of each tension element to produce the desired movement (pitch, yaw, and/or grip) at the wrist assembly 4500 and the end effector 4460. Specifically, the force transmission mechanism 4700 includes components that can be controlled to move some of the tension elements in a proximal direction (i.e., to pull in certain tension elements) while simultaneously allowing other tension elements to move in a distal direction (i.e., releasing or “paying out”). In this manner, the force transmission mechanism 4700 can cause the desired movement while also maintaining the desired tension within the tension elements. As shown in FIG. 7, the force transmission mechanism 4700 includes a set of drive components such as capstans 4710 and 4720 that rotate or “wind” a proximal portion of any of the tension elements to produce the desired tension element movement. In some embodiments, two proximal ends of a tension element, which are associated with opposing directions of a single degree of freedom, are connected to two independent drive capstans 4710 and 4720. This arrangement, which is generally referred to as an antagonist drive system, allows for independent control of the movement of (e.g., pulling in or paying out) each of the ends of the tension elements. The force transmission mechanism 4700 produces movement of the tension elements, which operates to produce the desired articulation movements (pitch, yaw, and/or grip) at the wrist assembly 4500 and end effector 4460. Accordingly, the force transmission mechanism 4700 includes components and controls to move a first proximal end portion of the tension element via the first capstan 4710 in a first direction (e.g., a proximal direction) and to move a second proximal end portion of the tension element via the second capstan 4720 in a second opposite direction (e.g., a distal direction). The force transmission mechanism 4700 can also move both proximal end portions of the tension element in the same direction. In this manner, the force transmission mechanism 4700 can maintain the desired tension within the tension elements.

In some embodiments, the force transmission mechanism 4700 can include any of the assemblies or components described in U.S. Provisional Patent Application Ser. No. 63/233,904, entitled “Surgical Instrument Cable Control and Routing Structures,” the disclosure of which is incorporated herein by reference in its entirety. In other embodiments, however, any of the medical devices described herein can have the two ends of a tension elements wrapped about a single capstan. This alternative arrangement, which is generally referred to as a self-antagonist drive system, operates the two ends of the tension element using a single drive motor.

Moreover, although the force transmission mechanism 4700 is shown as including capstans, in other embodiments, a force transmission mechanism can include one or more linear actuators that produce translation (linear motion) of a portion of the cables. Such proximal force transmission mechanisms can include, for example, a gimbal, a lever, or any other suitable mechanism to directly pull (or release) an end portion of any of the cables. For example, in some embodiments, the proximal force transmission mechanism 4700 can include any of the proximal mechanical structures or components described in U.S. Patent Application Pub. No. US 2015/0047454 A1 (filed Aug. 15, 2014), entitled “Lever Actuated Gimbal Plate,” or U.S. Pat. No. 6,817,974 B2 (filed Jun. 28, 2001), entitled “Surgical Tool Having Positively Positionable Tendon-Actuated Multi-Disk Wrist Joint,” each of which is incorporated herein by reference in its entirety.

The shaft 4410 can be any suitable elongated shaft that is coupled to the wrist assembly 4500 and to the force transmission mechanism 4700. Specifically, the shaft 4410 includes a proximal end portion 4411 that is coupled to the force transmission mechanism 4700, and a distal end portion 4412 that is coupled to the wrist assembly 4500 (e.g., coupled to a proximal wrist link of the wrist assembly 4500). The shaft 4410 defines a passageway or series of passageways through which the tension elements and other components (e.g., electrical wires, ground wires, or the like) can be routed from the force transmission mechanism 4700 to the wrist assembly 4500. In some embodiments, the shaft 4410 can be formed, at least in part with, for example, an electrically conductive material such as stainless steel. In such embodiments, the shaft 4410 may include any of an inner insulative cover or an outer insulative cover. Thus, the shaft 4410 can be a shaft assembly that includes multiple different components. For example, the shaft 4410 can include (or be coupled to) a spacer (not shown) that provides the desired fluid seals, electrical isolation features, and any other desired components for coupling the wrist assembly 4500 to the shaft 4410. Similarly stated, although the wrist assembly 4500 (and other wrist assemblies or links described herein) are described as being coupled to the shaft 4410, it is understood that any of the wrist assemblies or links described herein can be coupled to the shaft via any suitable intermediate structure, such as a spacer, a cable guide, additional joints and/or links, or the like. In some embodiments, the shaft 4410 may be a substantially rigid member, while in other embodiments, the shaft 4410 may be a flexible member. In some embodiments, any of the medical devices described herein can include any of the spacers, seals, cable guides, or other structure as described in U.S. Provisional Patent Application Ser. No. 63/319,926, filed on Feb. 15, 2022, and entitled “Surgical Instrument Including Electrical and Fluid Isolation Features,” the disclosure of which is incorporated herein by reference in its entirety.

The end effector 4460 includes a first tool member 4462 and a second tool member 4482, which in this embodiment are a pair of jaws, as shown in FIGS. 7-21. In alternative embodiments, the end effector 4460 can include other types of tool members such as a cautery device, a cutting device, or other medical tool. The first tool member 4462 (also referred to herein as “first jaw”) and the second tool member 4482 (also referred to herein as “second jaw”) can each rotate about the tool member rotation axis A1.

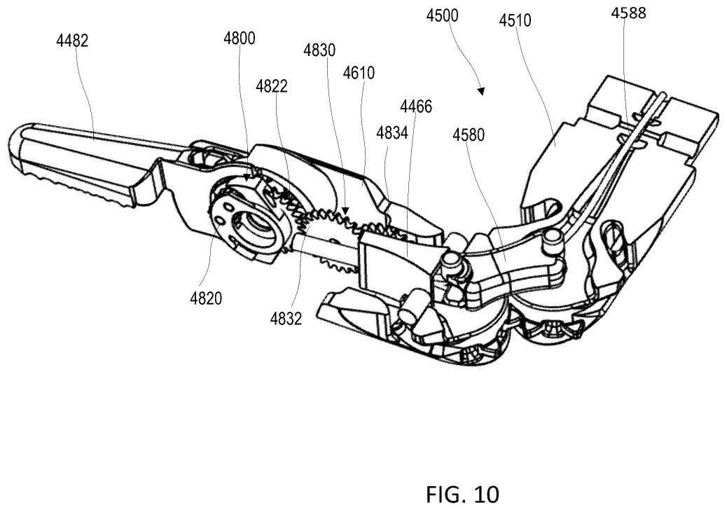

Referring to FIGS. 8-10, the wrist assembly 4500 includes a proximal wrist link 4510, a distal wrist link 4610, and a connector link 4580 (see, e.g., FIGS. 9 and 10). The proximal wrist link 4510 has a proximal end portion 4511 and a distal end portion 4512. The distal wrist link 4610 has a proximal end portion 4611 and a distal end portion 4612. As described in detail herein, the connector link 4580 is coupled between the proximal wrist link 4510 and the distal wrist link 4610 to form the articulating wrist assembly 4500. The proximal wrist link 4510 is coupled to the connector link 4580 via a pinned joint such that the connector link 4580 is rotatable with reference to the proximal wrist link 4510 about the third rotation axis A3. The distal wrist link 4610 is also coupled to the connector link 4580 via a pinned joint such that the distal wrist link 4610 is rotatable with reference to the connector link 4580 about the second rotation axis A2 and is also rotatable relative to the proximal wrist link 4510 about the third rotation axis A3. In this manner, the connector link 4580 maintains the coupling between the proximal wrist link 4510 and the distal wrist link 4610 during rotation of the distal wrist link 4610 relative to the proximal wrist link 4510. As shown, for example, in FIGS. 9 and 10, the distal wrist link 4610 and the proximal wrist link 4510 can be connected in rolling contact with gear teeth. This forces the angle of a centerline of the connector link 4580 to a centerline of the distal wrist link 4610 to be at all times the same as or substantially the same as the angle between the centerline of the connector link 4580 to the centerline of the proximal wrist link 4510. In some embodiments, the proximal wrist link 4510 is fixedly coupled to the shaft 4410 such that the proximal wrist link 4510 is not rotatable relative to the shaft. In other embodiments, however, the proximal wrist link 4510 can be rotatably coupled to the shaft 4410.

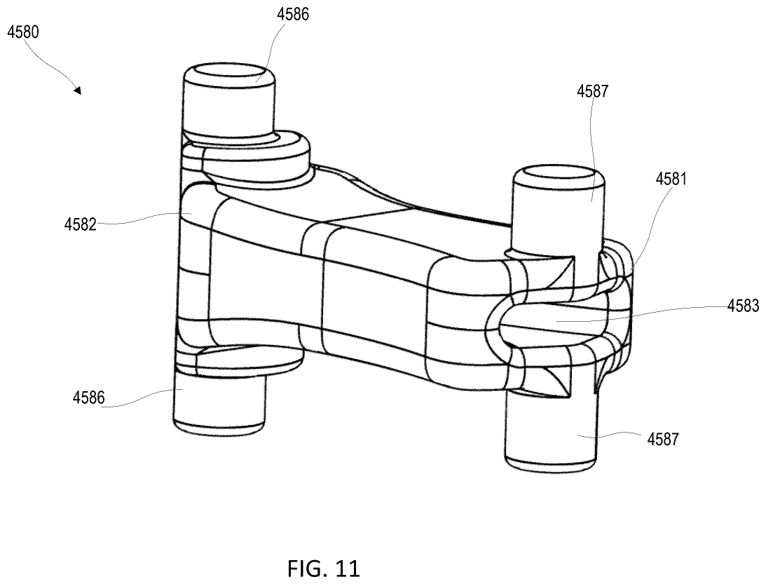

As shown, for example, in FIG. 11, the connector link 4580 includes a proximal end 4581 and a distal end 4582 and defines an internal pathway 4583 through which one or more elongate members can extend. The elongate members can be, for example, an electrical wire, a cable, or a position sensor such as, for example, a shape fiber described herein. In some embodiments, as shown in FIGS. 12-16, the internal pathway 4583 includes a tapered entry at both a proximal end of the internal pathway 4583 and a distal end of the internal pathway 4583. The tapered entries can prevent a sharp bend in the shape fiber 4588 thus maintain a desired minimum bend radius on the shape fiber 4588. In some embodiments, a distal end 4582 of the connector link 4580 is disposed distally of the distal link rotation axis A2. The connector link 4580 also includes protrusions 4587 and 4586 that are used to rotatably couple the connector link 4580 to the proximal wrist link 4510 and the distal wrist link 4610, respectively. For example, the protrusions 4587 and 4586 can be received respectively within corresponding openings (not shown) in the links 4510 and 4610. In other embodiments, the connector link 4580 may include openings and the links 4510, 4610 may include corresponding protrusions that can be received within the openings of the connector link 4580.

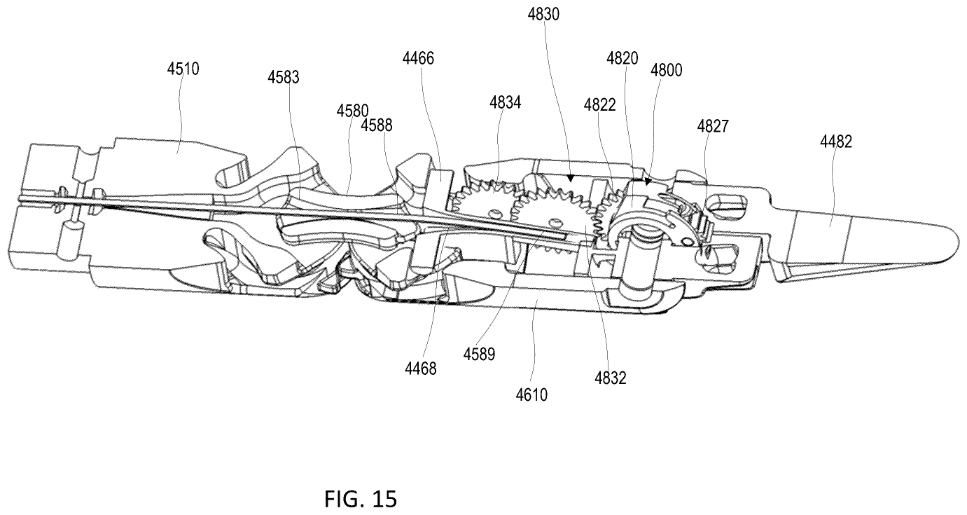

As shown in FIGS. 12-20, the wrist assembly 4500 also includes a differential 4800, a gear train 4830, a shape fiber carrier 4466, and a shape fiber 4588. The shape fiber 4588 can be a single optical fiber or a bundle of multiple optical fibers. The shape fiber carrier 4466 includes a shape fiber lumen 4468 and the shape fiber 4588 extends distally within the shape fiber lumen 4468 (see FIG. 15). More specifically, the shape fiber 4588 has a straight distal end portion 4589 (see FIG. 15) that is fixedly coupled to the shape fiber carrier 4466 within the shape fiber lumen 4468. The distal end portion 4589 has a desired length and facilitates accurate measurement of bends in the other portions of the shape fiber 4588. For example, the straight distal end portion 4589 allows for accurate transmission and reflection of optical signals for position and orientation determination. The straight distal end portion 4589 can have a predetermined length of (e.g., of at least 3 mm, between 3 mm and 6 mm), and the shape fiber lumen 4468 includes a distal portion that is shaped to accommodate the straight distal end portion 4589. In some embodiments, the shape fiber 4588 can have a minimum bend radius of, for example, between 5 mm and 6 mm. The shape fiber 4588 extends through the proximal wrist link 4510, through the internal pathway 4583 of the connector link 4580 and distally within the shape fiber lumen 4468.

The shape fiber carrier 4466 is pivotably coupled to the distal wrist link 4610 about a carrier axis C1 shown in FIG. 12, which is offset or spaced from the tool member axis A1. Thus, the distal end portion 4589 of the shape fiber 4588 is positioned between the carrier axis C1 and the tool member axis A1. The shape fiber carrier 4466 is operatively coupled to the tool members 4462 and 4482 via the differential 4800 and gear train 4830. Thus, the shape fiber carrier 4466 and the shape fiber 4588 are indirectly coupled to the first and second tool members 4462, 4482. In other words, the shape fiber carrier 4466 and the shape fiber 4588 are spaced apart from each of the first tool member 4462 and the second tool member 4482. The differential 4800 allows for the first tool member 4462 and the second tool member 4482 to rotate independently of each other while also being coupled to (and producing movement in) the shape fiber carrier 4466. As described above for previous embodiments, the shape fiber 4588 is coupled to the shape fiber carrier 4466 such that movement of the shape fiber carrier 4466 causes the shape fiber 4588 to bend or move. Because the shape fiber carrier 4466 is operatively coupled to the first tool member 4462 and the second tool member 4482 via the differential 4800 and gear train 4830, when the first tool member 4462 or the second tool member 4482 rotate relative to each other, the movement causes the shape fiber carrier 4466 to move, and the shape fiber 4588 coupled thereto to also move and thereby produce a signal related to the movement of the first tool member 4462, the second tool member 4482, or both. In some embodiments, the signal produced by the shape fiber 4588 includes a signal associated with an average of a first yaw angle of the first tool member 4462 and a second yaw angle of the second tool member 4482.

The gear train 4830 includes a proximal gear 4831, a distal gear 4832, and two middle gears 4833 and 4834. The shape fiber carrier 4466 includes a spur gear 4828 that is coupled to and engages the proximal gear 4831. The distal gear 4832 is coupled to and engages an integral ring gear 4822 of the differential 4800 to transfer motion from the differential 4800 to the shape fiber carrier 4466, described in more detail below. Middle gear 4833 couples distal gear 4832 to middle gear 4834 and middle gear 4834 in turn couples to proximal gear 4831. The gears of the gear train 4830 are shown by way of example and a different number of gears or gear configuration may be present in alternative embodiments, for example with a greater number or a fewer number of gears.

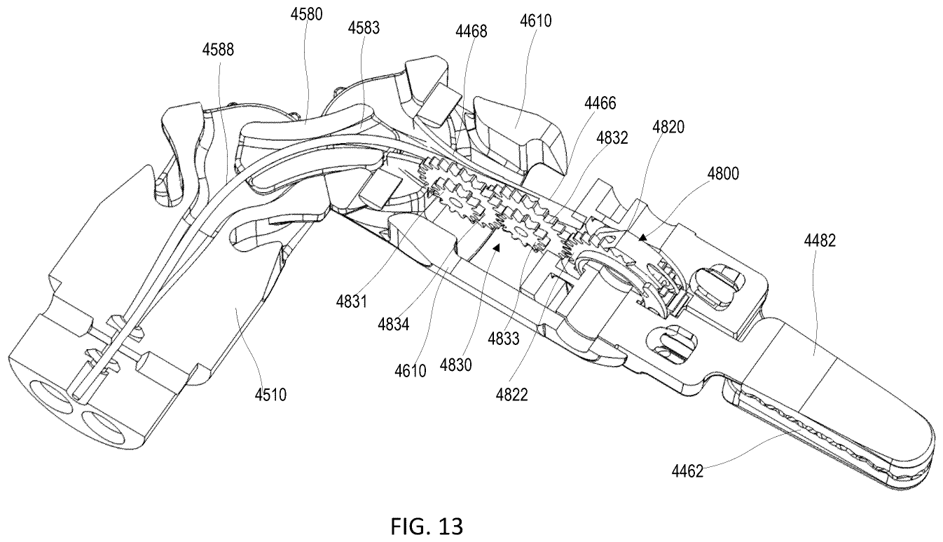

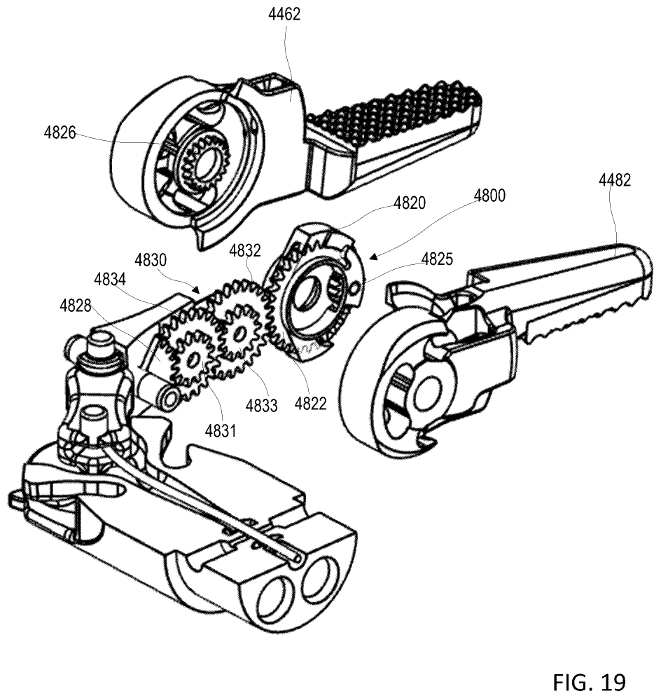

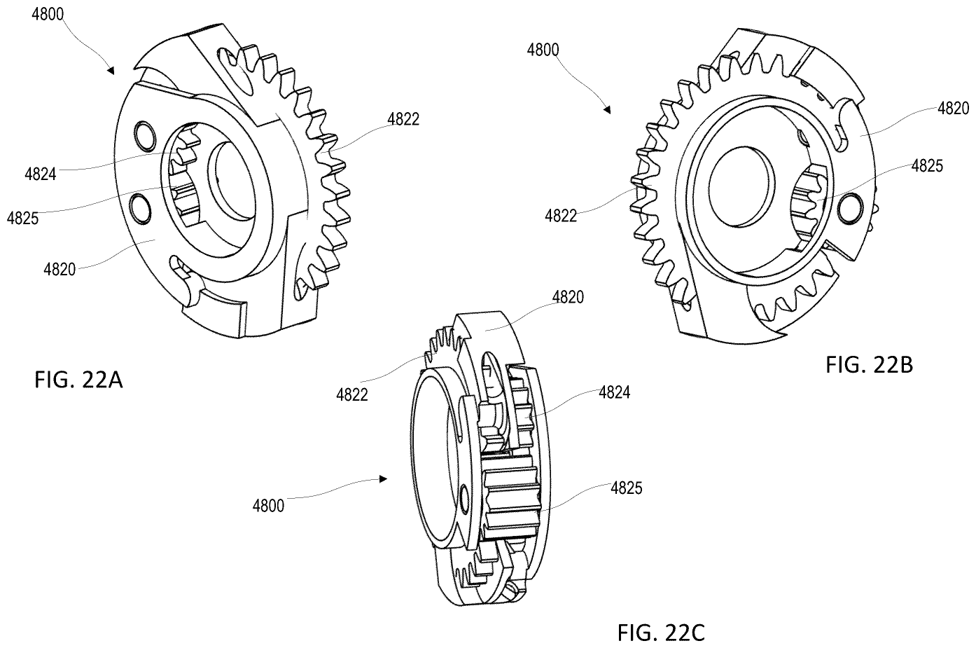

The differential 4800 is coupled to the first tool member 4462 and the second tool member 4482, and to the shape fiber carrier 4466 (via the gear train 4830). More specifically, the differential 4800 includes a housing 4820 with the integral ring gear 4822 on an exterior portion of the housing 4820 as shown, for example, in FIGS. 13, 15, 17 and 19-22C. As described above, the ring gear 4822 matingly engages the distal gear 4832 of the gear train 4830. The differential 4800 includes a first planet gear 4824 and a second planet gear 4825 that are disposed within the housing 4820 (see FIGS. 19-22C). The planet gears 4824, 4825 are each rotatably coupled to the housing 4820 and are rotatable relative to the housing 4820 about their own respective axes. The first planet gear 4824 is coupled to and matingly engages a first sun gear 4826 that is integral with the first tool member 4462, and the second planet gear 4825 is coupled to and matingly engages a second sun gear 4827 that is integral with the second tool member 4482 (see FIGS. 19-21). The first planet gear 4824 and the second planet gear 4825 also engage each other within the housing 4820 (see FIGS. 22A-22C).

When one of the first tool member 4462 or the second tool member 4482 rotates relative to the other such that a direction of the average of the positions of the tool members 4462, 4482 changes (e.g., with respect to the distal wrist link 4610), the associated sun gear 4826 or 4827 of the respective tool member 4462 or 4482 will engage the corresponding planet gear 4824 or 4825 of the differential 4800, which in turn causes the housing 4820 to rotate about the axis A1. The rotation of the housing 4820 causes the gear train 4830 to rotate via the engagement of the ring gear 4822 with the distal gear 4832 of the gear train 4830. The gear train 4830 will then cause movement of the shape fiber carrier 4466 via the engagement between the proximal gear 4831 of the gear train 4830 and the spur gear 4828 of the shape fiber carrier 4466. Specifically, the shape fiber carrier 4466 rotates relative to the distal wrist link 4610 about the rotation axis C1. This in turn causes the shape fiber 4588 to move.

To further illustrate the rotation of the housing 4820, an example will be described where the first tool member 4462 rotates relative to the second tool member 4482. For example, consider the case where the first tool member 4462 rotates about axis A1 via first tension elements relative to the second tool member 4482 with the second tool member 4482 being held fixed about axis A1 by second tension elements. In such an example, the rotation of the first tool member 4462 about axis A1 causes the first sun gear 4826 (integral to the first tool member 4462) to rotate about the axis A1, which then causes the first planet gear 4824 (rotatably coupled to the first sun gear 4826) to rotate about the axis of the first planet gear 4824. Meanwhile, because the second tool member 4482 is held fixed, the second sun gear 4827 (integral to the second tool member 4482) is held rotationally fixed. The second planet gear 4825 (rotatably coupled to the second sun gear 4827) orbits around the second sun gear 4827 that is held fixed. Thus, the second planet gear 4827 rotates about its own axis as it orbits around the second sun gear 4827 of the second tool member 4482. The orbiting of the second planet gear 4825 (which is contained within the housing 4820) in turn causes the housing 4820 of the differential 4800 to rotate, thereby causing movement of the shape fiber carrier 4466 and the shape fiber 4588. In other examples, the housing 4820 will rotate when the second tool member 4482 rotates relative to the first tool member 4462, and also when both tool members 4462 and 4482 rotate together in yaw relative to the wrist assembly 4500.

When the direction of the average of the positions of the tool members 4462, 4482 changes, the differential housing 4820 will be rotated accordingly, which in turn moves the shape fiber 4588 operatively coupled thereto. The rotation or orientation of the housing 4820 is associated with an average of a first yaw angle of the first tool member 4462 and a second yaw angle of the second tool member 4482. More specifically, the measurement of the position of the tool members 4462, 4482 is determined as the shape fiber 4588 bends in proportion to the average/differential between the two tool members 4462, 4482. The use of the differential 4800 limits the motion of the shape fiber 4588 to accommodate the limits on the bend radius of the shape fiber 4588 and to accommodate the straight section at the distal tip of the shape fiber 4588. In some embodiments, the differential 4800 and/or the gear train 4830 are configured such that the ratio of the angle of rotation of the first tool member 4462 and the second tool member 4482 to the angle of rotation of the shape fiber carrier 4466 is greater than 1:1. In this manner, the differential 4800 and/or the gear train 4830 can reduce the amount of movement transmitted to the shape fiber carrier 4466 thereby accommodating space constraints within the medical device 4400. In some embodiments the ratio of movement of the tool members 4462, 4482 to the shape fiber carrier 4466 is about 2:1. In other embodiments, the ratio of movement of the tool members 4462, 4482 to the shape fiber carrier 4466 is about 3:1. For example, the rotational movement of the tool members 4462, 4482 may be each +/−110 degrees while the rotational movement of the shape fiber 4588 can be limited to +/−45 degrees relative to a centerline of the shape fiber carrier 4466. When the shape fiber 4588 moves or bends, the shape fiber 4588 produces a signal related to the movement of the shape fiber 4588. In some embodiments, the signal produced by the shape fiber 4588 is associated with an average of a first yaw angle of the first tool member 4462 and a second yaw angle of the second tool member 4482.

As described herein, the differential 4800 can allow for the use of a single shape fiber sensor to determine the position of the two tool members 4462 and 4482. The single shape fiber sensor (e.g., shape fiber 4588) can be used to determine position and orientation of the tool members 4462, 4482, with the shape fiber 4588 not being directly coupled to the tool members 4462, 4482. In addition to providing position and orientation information of the tool members 4462, 4482, in some embodiments, movement of the shape fiber carrier 4466 also causes the shape fiber 4588 to produce a signal related to movement of the distal wrist link 4610. In some embodiments, movement of the shape fiber carrier 4466 additionally causes the shape fiber 4588 to produce a signal related to movement of a proximal articulating joint and/or link such as the proximal wrist link 4510 and other flexible instrument portions (e.g., a flexible instrument shaft). Accordingly, the shape fiber 4588 may be used to determine position and orientation of the tool members 4462, 4482 as well as of other portions of the medical device.

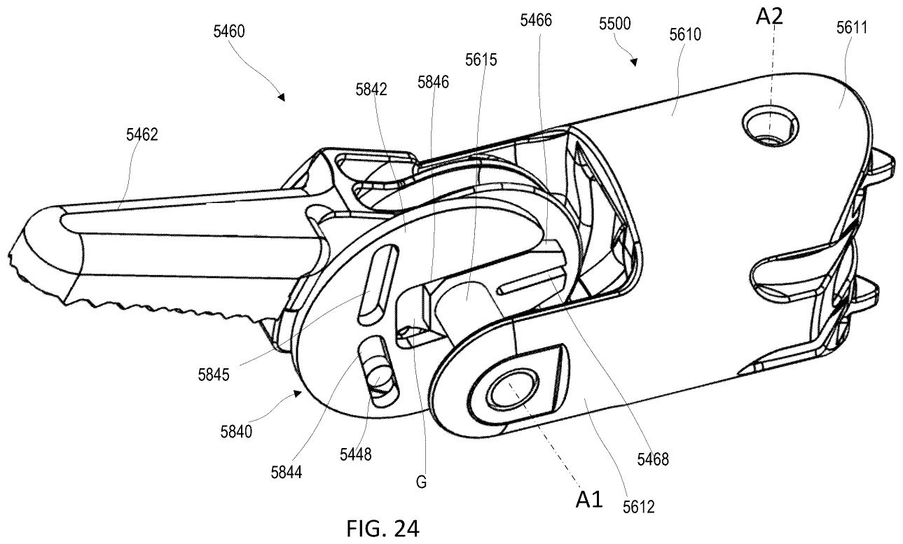

FIGS. 23-29E illustrate a portion of a medical device 5400, according to an embodiment. In some embodiments, the medical device 5400 or any of the components therein are optionally parts of an instrument for a surgical system that performs surgical procedures, and which surgical system can include a manipulator unit, a series of kinematic linkages, a series of cannulas, or the like. The medical device 5400 (and any of the instruments described herein) can be used in any suitable surgical system, such as the MIRS system 1000 shown and described above. The medical device 5400 includes a wrist assembly 5500, and an end effector 5460 including a first tool member 5462 and a second tool member 5482. The medical device 5400 can also include a shaft (not shown) and a force transmission mechanism (not shown) that can be the same as or similar to and function the same as or similar to the shaft 4410 and force transmission mechanism 4700 discussed above. The medical device 5400 also includes one or more tension elements 5420 (see FIGS. 23 and 24). The tension elements 5420 can be, for example, cables, bands or the like that couple the force transmission mechanism to the wrist assembly 5500 and end effector 5460.

As discussed above for previous embodiments, the medical device 5400 is configured such that movement of one or more of the tension elements 5420 produces rotation of the end effector 5460 about a first rotation axis A1 (also referred to herein as “tool member axis”), which functions as a yaw axis (the term yaw is arbitrary), rotation of the wrist assembly 5500 about a second rotation axis A2 (also referred to as “distal wrist rotation axis”) or a third rotation axis (not shown) (also referred to as “proximal wrist rotation axis”), a cutting (or gripping) rotation of the tool members of the end effector 5460 about the first rotation axis A1, or any combination of these movements. FIG. 23 illustrate the distal wrist link 5610 in a straight configuration and the tool members 5462 and 5482 in an open configuration (rotated spaced apart relative to each other). Changing the pitch and/or yaw of the medical device 5400 can be performed by manipulating the tenson elements 5420 in a similar manner as that described with reference to the device 2400 and 4400 described above and described in U.S. Provisional Patent Application Ser. No. 63/233,904, entitled “Surgical Instrument Cable Control and Routing Structures” and U.S. Patent Publication No. 2020/0390430, (filed Aug. 21, 2020), entitled “Low-Friction, Small Profile Medical Tools Having Easy-to-Assemble Components,” the disclosure of each of which is incorporated herein by reference above.

In this embodiment the first tool member 5462 and the second tool member 5482 are a pair of jaws, as shown, in FIGS. 23-29E. In alternative embodiments, the end effector 5460 can include other types of tool members such as a cautery device, a cutting device, or other medical tool. The first tool member 5462 (also referred to herein as “first jaw”) and the second tool member 5482 (also referred to herein as “second jaw”) can each rotate about the tool member rotation axis A1. More specifically, the first tool member 5462 and the second tool member 5482 are rotatably coupled to the distal wrist link 5610 via a pin 5615.

The wrist assembly 5500 includes a proximal wrist link (not shown) that can be the same as or similar to proximal wrist link 4510, a distal wrist link 5610, and optionally a connector link (not shown) that can be the same as or similar to the connector link 4580. The distal wrist link 5610 has a proximal end portion 5611 and a distal end portion 5612. The optional connector link can be coupled between the proximal wrist link and the distal wrist link 5610 to form the articulating wrist assembly 5500 in the same manner as described for wrist assembly 4500. In some embodiments, the proximal wrist link is fixedly coupled to the shaft such that the proximal wrist link is not rotatable relative to the shaft. In other embodiments, however, the proximal wrist link can be rotatably coupled to the shaft.

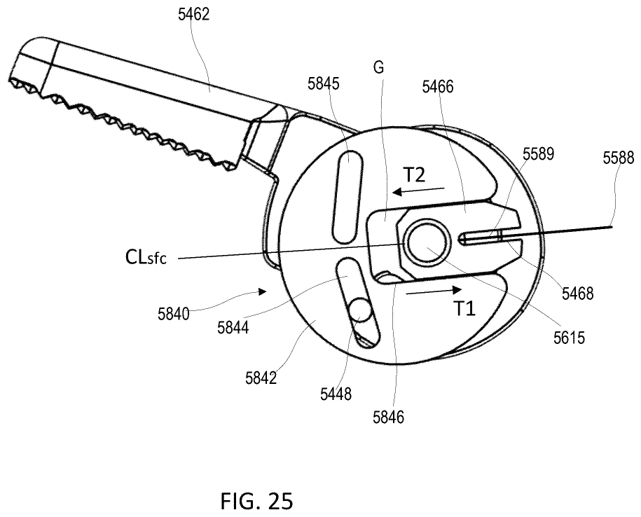

As shown, for example, in FIGS. 24-25, the wrist assembly 5500 also includes an intermediate coupling mechanism 5840, a shape fiber carrier 5466, and a shape fiber 5588. In this embodiment, the intermediate coupling mechanism functions as a cam mechanism 5840 described in more detail below. The shape fiber carrier 5466 is rotatably coupled to the first tool member 5462 and the second tool member 5482 via the pin 5615 such that the shape fiber carrier 5466 can rotate with the first jaw 5462 and/or the second jaw 5482 about the tool axis A1. The shape fiber carrier 5466 defines a shape fiber lumen 5468 in which a distal end of the shape fiber 5588 extends. Thus, the shape fiber 5588 is coupled to the shape fiber carrier 5466 such that movement of the shape fiber carrier 5466 causes the shape fiber 5588 to bend or move. As shown, for example, in FIGS. 24-27, the shape fiber lumen 5468 has a closed distal end. In alternative embodiments, the shape fiber lumen 5468 may extend through the shape fiber carrier 5466 to an open end, through an opening through the pin 5615, and extend outside and distal end of the shape fiber carrier 5466. Further, in some alternative embodiments, the shape fiber carrier 5466 and the pin 5615 can be formed integrally as a single component. In another alternative embodiment, the shape fiber carrier 5466 is formed integrally with the pin 5615 as a single component, and the shape fiber lumen 5468 extends through the shape fiber carrier 5466 to an open end, through an opening in the pin 5615, and extend outside and distal of the shape fiber carrier 5466, and the shape fiber carrier 5466 is formed integrally with the pin 5615 as a single component.

Because the shape fiber carrier 5466 is rotatably coupled to the first tool member 5462 and the second tool member 5482, when the first tool member 5462 or the second tool member 5482 rotates relative to each other, or are moved relative to the distal link 5610, the movement causes the shape fiber carrier 5466 to move, and, in turn, the shape fiber 5588 coupled thereto to move. The shape fiber 5588 thereby produces a signal related to the movement of the first tool member 5462, the second tool member 5482 or both as described in more detail below. In some embodiments, the signal produced by the shape fiber 5588 includes a signal associated with an average of a first yaw angle of the first tool member 5462 and a second yaw angle of the second tool member 5482.

The shape fiber 5588 can be a single optical fiber or a bundle of multiple optical fibers. The shape fiber 5588 has a straight distal end portion 5589 (see FIG. 25) that is fixedly coupled to the shape fiber carrier 5466 within the shape fiber lumen 5468 such that the distal end portion 5589 of the shape fiber 5588 is spaced apart (e.g., disposed at a distance) from each of the first tool member 5462 and the second tool member 5482. In other words, the shape fiber 5588 is indirectly coupled to the first tool member 5462 and the second tool member 5482. The distal end portion 5589 has a desired length and facilitates accurate measurement of bends in the other portions of the shape fiber 5588. For example, the straight distal end portion 5589 allows for accurate transmission and reflection of optical signals for position and orientation determination. The straight distal end portion 5589 can have a predetermined length (e.g., of at least 3 mm, between 3 mm and 6 mm), and the shape fiber lumen 5468 includes a distal portion that is shaped to accommodate the straight distal end portion 5589. In some embodiments, the shape fiber 5588 can have a minimum bend radius of, for example, between 4 mm to 5 mm. The shape fiber 5588 extends through the proximal wrist link (not shown), through the internal pathway of the connector link (not shown) and distally within the shape fiber lumen 5468.

The cam mechanism 5840 includes a cam disk 5842 that defines cam slots 5844 and 5845 and a notch 5846. The shape fiber carrier 5466 is received within the notch 5846 of the cam disk 5842 such that when the cam disk 5842 rotates, the shape fiber carrier 5466 coupled within the notch 5846 also rotates about the tool axis A1. Moreover, the cam disk 5842 rotates in response to the first tool member 5462 and/or second tool member 5482 rotating relative to each other and/or rotating relative to the distal link 5610. Accordingly, the cam disk 5842 functions as an intermediate coupling mechanism to couple the shape fiber carrier 5466 to the first and second tool members 5462, 5482. The cam disk 5842 can also slide or translate relative to the shape fiber carrier 5466 in the direction or arrows T1 and T2 parallel to a centerline CLsfc of the shape fiber carrier 5466 as shown in FIG. 25 and as described in more detail below. Thus, as described below, the cam mechanism can produce eccentric motion of the cam disk 5842.

As shown, for example, in FIGS. 25-27, the cam disk 5842 is positioned between the first tool member 5462 and the second tool member 5482. The first tool member 5462 has a cam pin 5448 that is received within the cam slot 5844 of the cam disk 5842 and the second tool member 5482 has a cam pin 5449 that is received within the cam slot 5845 of the cam disk 5842. As the first tool member 5462 rotates about the tool axis A1, the cam pin 5448 moves within the cam slot 5844, and when the second tool member 5482 rotates about the tool axis A1, the cam pin 5449 moves within the slot 5845. Thus, the rotational movement of the first tool member 5462 and the second tool member 5482 causes movement of the pins within the cam slots 5844 and 5845 of the cam disk 5842. This movement, in turn, causes the cam disk 5842 to rotate. The cam slots 5844 and 5855 are linear and slightly angled relative to a centerline CLsfc of the shape fiber carrier 5466, and are sized and positioned on the cam disk 5842 to provide for some minimal interference between the cam disk 5842 and the tool members 5462 and 5482, but not too much interference so as to negatively interfere with the movement of the tool members 5462 and 5482. If the cam slots 5844 and 5845 were positioned to extend orthogonal to the centerline CLsfc of the shape fiber carrier 5466, the range of motion of the tool members 5462 and 5482 would be too limited (e.g., restricted to less than a desired available range of motion of the tool members 5462, 5482) and there would be poor engagement between the cam pins 5448 and 5489 and the cam disk 5842. Instead, it is desirable for the cam slots 5844 and 5845 to be orthogonal, or as close to orthogonal as possible to the travel path of the cam pins 5448 and 5449 as the tool members 5462, 5428 move between fully open and closed configurations. Thus, the location of the cam slots 5844 and 5845 relative to the location and movement of the cam pins 5448, 5489 is important for proper functioning of the cam mechanism 5840.

Although the cam slots 5844 and 5845 are shown disposed on the cam disk 5842 in the same plane as the shape fiber carrier 5466, in alternative embodiments, the cam slots can be offset from the shape fiber carrier 5466. In some alternative embodiments, the shape fiber carrier 5466 can be positioned distal of the cam slots 5844 and 5845 rather than proximal as in the medical device 5400.

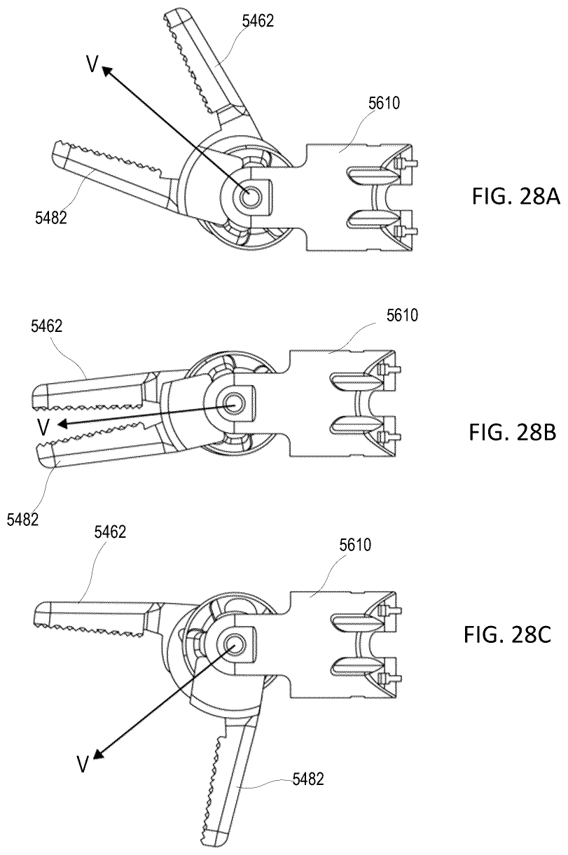

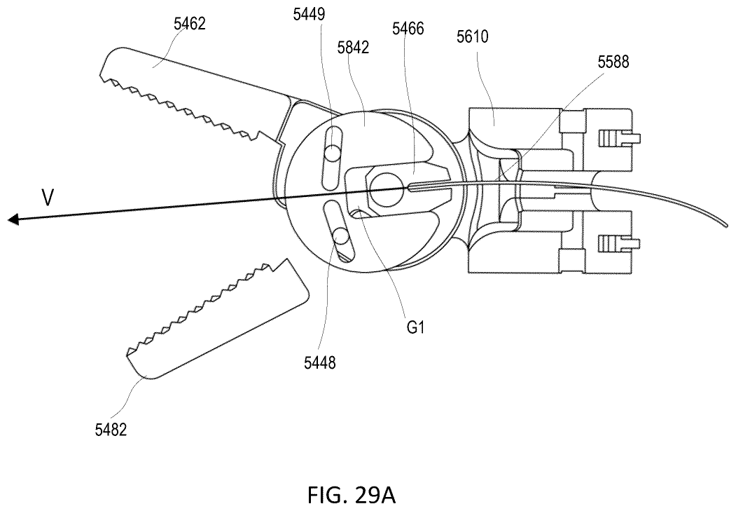

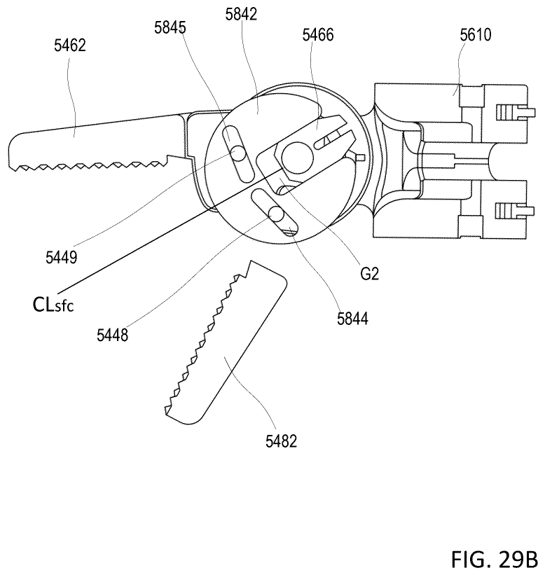

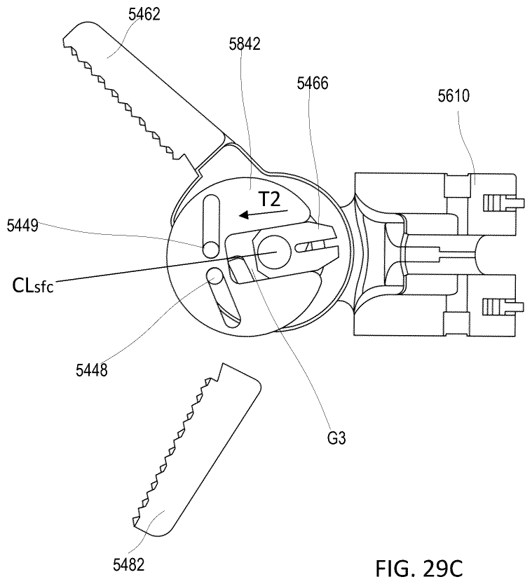

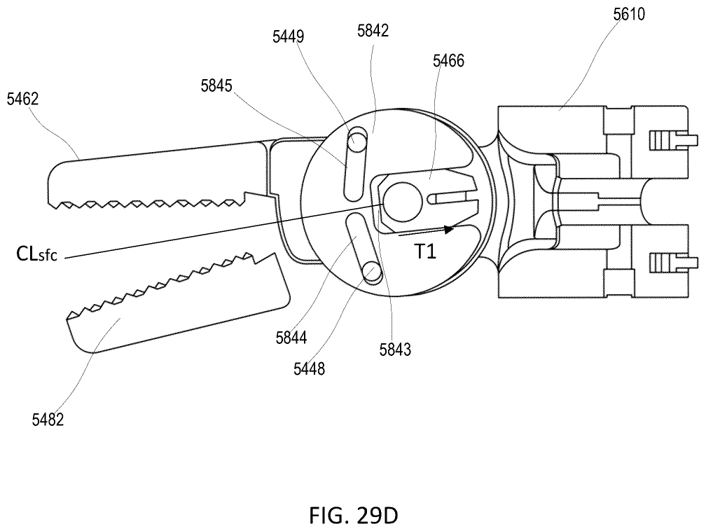

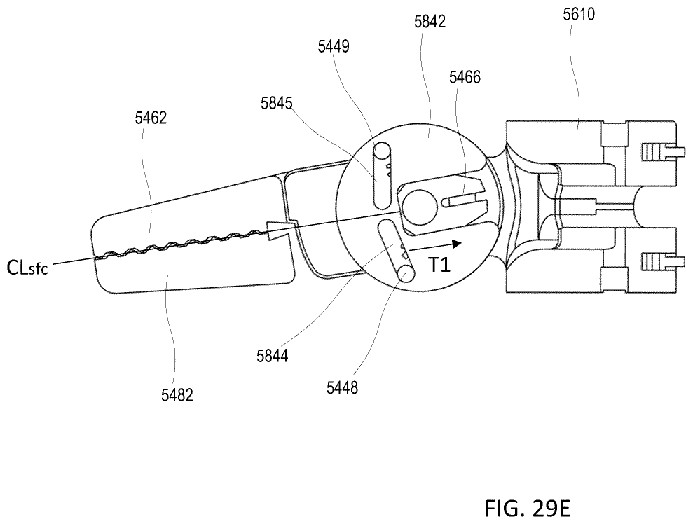

As described above, the length and position of the cam slots 5844, 5845 and the movement of the cam disk 5842 relative to the shape fiber carrier 5466 provides for the shape fiber carrier 5466, and the shape fiber 5588 coupled thereto, to be maintained at a center position between the first tool member 5462 and the second tool member 5482 throughout the rotational movement of the first tool member 5462 and the second tool member 5482. Similarly stated, the rotational position of the cam disk 5842 is associated with the average of the first yaw angle of the first tool member 5462 and the second yaw angle of the second tool member 5482. More specifically, as shown in FIGS. 28A-29A, a shape fiber vector V is defined at the centerline CLsfc of the shape fiber carrier 5466. As the first tool member 5462 and the second tool member 5482 are rotated relative to the distal link 5610 (FIG. 28A) and/or to each other (FIGS. 28B and 28C), the centerline CLsfc of the shape fiber carrier 5466 is maintained centered between the first tool member 5462 and the second tool member 5482. Thus, the distal end 5589 of the shape fiber 5588 (coupled to the shape fiber carrier 5466) is oriented along the vector V associated with the average of the first yaw angle of the first tool member 5462 and the second yaw angle of the second tool member 5482, and outputs a signal associated with the vector V.

FIGS. 29A-29E illustrate example positions of the first tool member 5462 and the second tool member 5482 and the position and orientation of the cam disk 5842 relative to the shape fiber carrier 5466 during rotational movement of the first tool member 5462 and the second tool member 5482 relative to each other and/or rotated relative to the distal link 5610. A portion of the second tool member 5482 is not shown for illustration purposes. FIG. 29A illustrates the first tool member 5462 and the second tool member 5482 rotated apart from each other at a first orientation. In this orientation, a gap G1 is defined between the cam disk 5842 and the shape fiber carrier 5466. FIG. 29B illustrates the first tool member 5462 and the second tool member 5482 rotated apart from each other at a second orientation wherein the jaws are spaced slightly further apart than in the first orientation and are also rotated downward relative to the distal link 5610 about the axis A1. In this orientation, a gap G2 is defined between the cam disk 5842 and the shape fiber carrier 5466 that is substantially the same as the gap G1 and the cam pins 5448 and 5449 are positioned approximately at a center of the corresponding cam slots 5844 and 5845. FIG. 29C illustrates the first tool member 5462 and the second tool member 5482 rotated apart from each other at a maximum distance (note that the pins 5448 and 5449 are at the maximum travel distances within the cam slots 5844 and 5845). In this orientation, a gap G3 is defined between the cam disk 5842 and the shape fiber carrier 5466 that is larger than the gap G1 and the gap G2. More specifically, as the first tool member 5462 and the second tool member 5482 are moved further apart from each other, the cam pin 5448 moves to a position at a top of the cam slot 5844, the cam pin 5449 moves to a position at a bottom of the cam slot 5845, and the cam disk 582 translates in the direction of T2 relative to the shape fiber carrier 5466. Thus, the cam disk 5842 both rotates and translates.

FIG. 29D illustrates the first tool member 5462 and the second tool member 5482 in a partially closed orientation relative to each other (i.e., rotated toward each other) and slightly rotated relative to the distal link 5610. As the first tool member 5462 and the second tool member 5482 are moved closer together, the cam pin 5448 moves to a position close to the bottom of the cam slot 5844, the cam pin 5449 moves to a position close to the top of the cam slot 5845, and the cam disk 5842 slides in the direction T1 relative to the shape fiber carrier 5466 to define a gap G4 between the shape fiber carrier 5466 and the cam disk 5842 that is smaller than the gaps G1, G2 and G3. FIG. 29E illustrates the first tool member 5462 and the second tool member 5482 in a fully closed orientation relative to each other. In this position the cam pin 5448 moves to a position at the bottom of the cam slot 5844, the cam pin 5449 moves to a position at the top of the cam slot 5845, and the cam disk 5842 slides in the direction T1 relative to the shape fiber carrier 5466 and closes the gap between the shape fiber carrier 5466 and the cam disk 5842.

When one of the first tool member 5462 or the second tool member 5482 rotates relative to the other such that a direction of the average of the positions of the tool members 5462, 5482 changes (e.g., with respect to the distal wrist link 5610), the shape fiber carrier 5466 will move as a result of the engagement between the cam mechanism 5840 and the first tool member 5462 and the second tool member 5482. As shown, for example, in FIGS. 29A-29E, the shape fiber carrier 5466 rotates relative to the distal wrist link 5610 about the tool axis A1. This in turn causes the shape fiber 5588 to move. The rotation or orientation of the shape fiber carrier 5466 is associated with an average of a first yaw angle of the first tool member 5462 and a second yaw angle of the second tool member 5482 (illustrated as the vector V in FIGS. 28A-29A). More specifically, the measurement of the position of the tool members 5462, 5482 is determined as the shape fiber 5588 bends in proportion to the average between the two tool members 5462, 5482. The use of the cam mechanism 5840 can limit the range of motion of first tool member 5462 and the second tool member 5482, and/or the range of motion of the shape fiber 5588 to accommodate the limits on the bend radius of the shape fiber 5588 and to accommodate the straight section at the distal tip portion 5589 of the shape fiber 5588. In some embodiments, the signal produced by the shape fiber 5588 is associated with an average of a first yaw angle of the first tool member 5462 and a second yaw angle of the second tool member 5482.

As described herein, the cam mechanism 5840 can allow for the use of a single shape fiber sensor (e.g., shape fiber 5588) to determine the position of the two tool members 5462 and 5482. The single shape fiber sensor can be used to determine position and orientation of the tool members 5462, 5482, with the shape fiber 5588 not being directly coupled to the tool members 5462, 5482. In addition to providing position and orientation information of the tool members 5462, 5482, in some embodiments, movement of the shape fiber carrier 5466 also causes the shape fiber to produce a signal related to movement of the distal wrist link 5610. In some embodiments, movement of the shape fiber carrier 5466 additionally causes the shape fiber 5588 to produce a signal related to movement of a proximal articulating joint and/or link such as the proximal wrist link and other flexible instrument portions (e.g., a flexible instrument shaft). Accordingly, the shape fiber 5588 may be used to determine position and orientation of the tool members 5462, 5482 as well as of other portions of the medical device.

FIGS. 30-38 illustrate a portion of a medical device 6400 (and portions thereof), according to an embodiment. In some embodiments, the medical device 6400 or any of the components therein are optionally parts of an instrument for a surgical system that performs surgical procedures, and which surgical system can include a manipulator unit, a series of kinematic linkages, a series of cannulas, or the like. The medical device 6400 (and any of the instruments described herein) can be used in any suitable surgical system, such as the MIRS system 1000 shown and described above. The medical device 6400 includes a wrist assembly 6500, and an end effector 6460 including a first tool member 6462 and a second tool member 6482. The medical device 6400 can also include a shaft (not shown) and a force transmission mechanism (not shown) that can be the same as or similar to and function the same as or similar to the shaft 4410 and force transmission mechanism 4700 discussed above. The medical device 6400 also includes one or more tension elements (not shown) such as tension elements 5420 described above. The tension elements can be, for example, cables, bands or the like that couple the force transmission mechanism to the wrist assembly 6500 and end effector 6460.

As discussed above for previous embodiments, the medical device 6400 is configured such that movement of one or more of the tension elements produces rotation of the end effector 6460 about a first rotation axis A1 (also referred to herein as “tool member axis”), which functions as a yaw axis (the term yaw is arbitrary), rotation of the wrist assembly 5500 about a second rotation axis A2 (also referred to as “distal wrist rotation axis”) or a third rotation axis (not shown) (also referred to as “proximal wrist rotation axis”), a cutting (or gripping) rotation of the tool members of the end effector 6460 about the first rotation axis A1, or any combination of these movements. FIG. 30 illustrate the distal wrist link 6610 in a straight configuration and the tool members 6462 and 6482 in a closed configuration relative to each other. Changing the pitch or yaw of the medical device 6400 can be performed by manipulating the tenson elements in a similar manner as that described with reference to the device 2400 and 4400 described above and described in U.S. Provisional Patent Application Ser. No. 63/233,904, entitled “Surgical Instrument Cable Control and Routing Structures” and U.S. Patent Publication No. 2020/0390430, (filed Aug. 21, 2020), entitled “Low-Friction, Small Profile Medical Tools Having Easy-to-Assemble Components,” the disclosure of each of which is incorporated herein by reference above.

In this embodiment the first tool member 6462 and the second tool member 6482 are a pair of jaws, as shown, in FIGS. 30-35 and 37. In alternative embodiments, the end effector 6460 can include other types of tool members such as a cautery device, a cutting device, or other medical tool. The first tool member 6462 (also referred to herein as “first jaw”) and the second tool member 6482 (also referred to herein as “second jaw”) can each rotate about the tool member rotation axis A1 (see FIG. 30). More specifically, the first tool member 6462 and the second tool member 6482 are rotatably coupled to the distal wrist link 6610 via a pin 6615.

The wrist assembly 6500 includes a proximal wrist link (not shown) that can be the same as or similar to proximal wrist link 4510, a distal wrist link 6610, and optionally a connector link (not shown) that can be the same as or similar to the connector link 4580. The distal wrist link 6610 has a proximal end portion 6611 and a distal end portion 6612. The optional connector link is coupled between the proximal wrist link and the distal wrist link 6610 to form the articulating wrist assembly 6500 in the same manner as described for wrist assembly 4500. In some embodiments, the proximal wrist link is fixedly coupled to the shaft such that the proximal wrist link is not rotatable relative to the shaft. In other embodiments, however, the proximal wrist link can be rotatably coupled to the shaft.

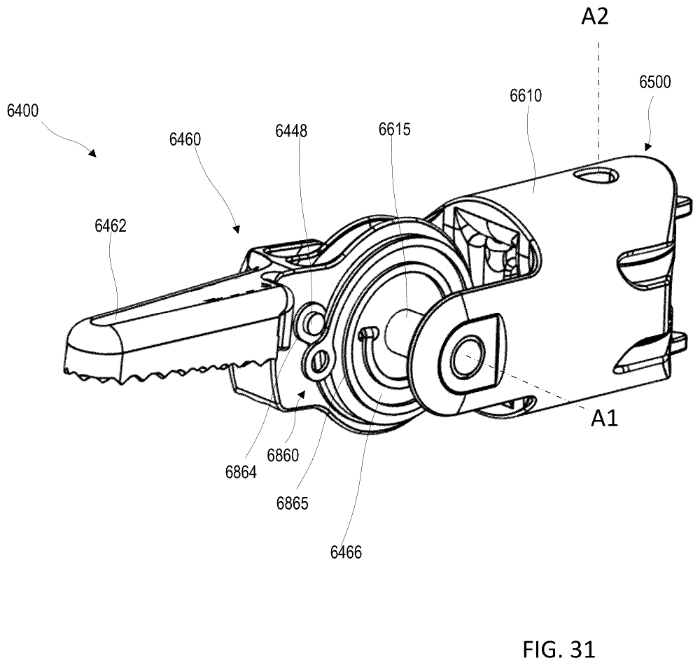

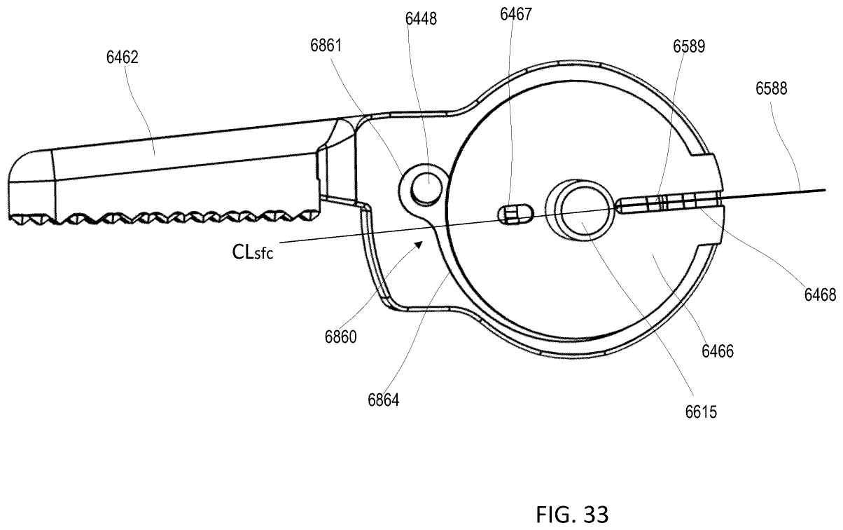

As shown, for example, in FIGS. 31-37, the wrist assembly 6500 also includes an intermediate coupling mechanism 6860, a shape fiber carrier 6466, and a shape fiber 6588 (see FIG. 33). In this embodiment, the intermediate coupling mechanism 6860 is a torsion spring mechanism that includes a first spring 6864 and a second spring 6865 and is described in more detail below. The shape fiber carrier 6466 is coupled to the first tool member 6462 and the second tool member 6482 via the pin 6615 such that the shape fiber carrier 6466 can rotate with the first jaw 6462 and/or the second jaw 6482 about the tool axis A1. The shape fiber carrier 6466 defines a shape fiber lumen 6468 in which a distal end of the shape fiber 6588 extends (see FIG. 33). Thus, the shape fiber 6588 is coupled to the shape fiber carrier 6466 such that movement of the shape fiber carrier 6466 causes the shape fiber 6588 to bend or move. The shape fiber carrier 6466 also defines an opening 6467 to which springs of the torsion spring mechanism 6860 are coupled as described below.

Because the shape fiber carrier 6466 is rotatably coupled to the first tool member 6462 and the second tool member 6482, when the first tool member 6462 or the second tool member 6482 rotates relative to each other, or are moved relative to the distal link 6610, the movement causes the shape fiber carrier 6466 to move, and, in turn, the shape fiber 6588 coupled thereto to move. The shape fiber 6588 thereby produces a signal related to the movement of the first tool member 6462, the second tool member 6482, or both, as described in more detail below. In some embodiments, the signal produced by the shape fiber 6588 includes a signal associated with an average of a first yaw angle of the first tool member 6462 and a second yaw angle of the second tool member 6482.

The shape fiber 6588 can be a single optical fiber or a bundle of multiple optical fibers. The shape fiber 6588 has a straight distal end portion 6589 (see FIG. 33) that is fixedly coupled to the shape fiber carrier 6466 within the shape fiber lumen 5468 such that the distal end portion 6589 of the shape fiber 6588 is spaced apart (e.g., disposed at a distance) from each of the first tool member 6462 and the second tool member 6482. In other words, the shape fiber 5588 is indirectly coupled to the first tool member 6462 and the second tool member 6482. The distal end portion 6589 has a desired length and facilitates accurate measurement of bends in the other portions of the shape fiber 6588. For example, the straight distal end portion 6589 allows for accurate transmission and reflection of optical signals for position and orientation determination. The straight distal end portion 6589 can have a length of at least 3 mm, for example between 3 mm and 6 mm, and the shape fiber lumen 6468 includes a distal portion that is shaped to accommodate the straight distal end portion 5589. In some embodiments, the shape fiber 6588 can have a minimum bend radius of, for example, between 4 mm to 5 mm. The shape fiber 6588 extends through the proximal wrist link (not shown), through the internal pathway of the connector link (not shown) and distally within the shape fiber lumen 6468.

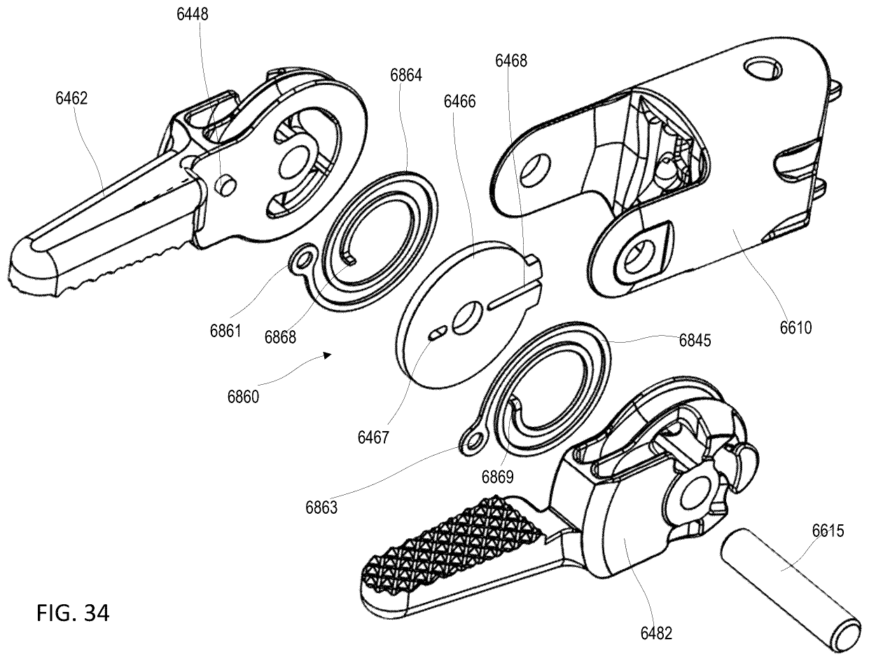

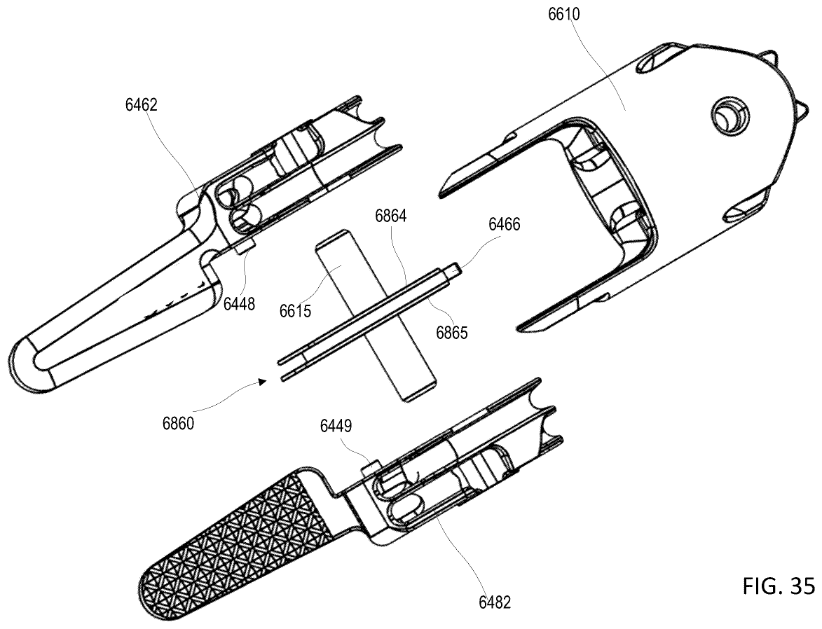

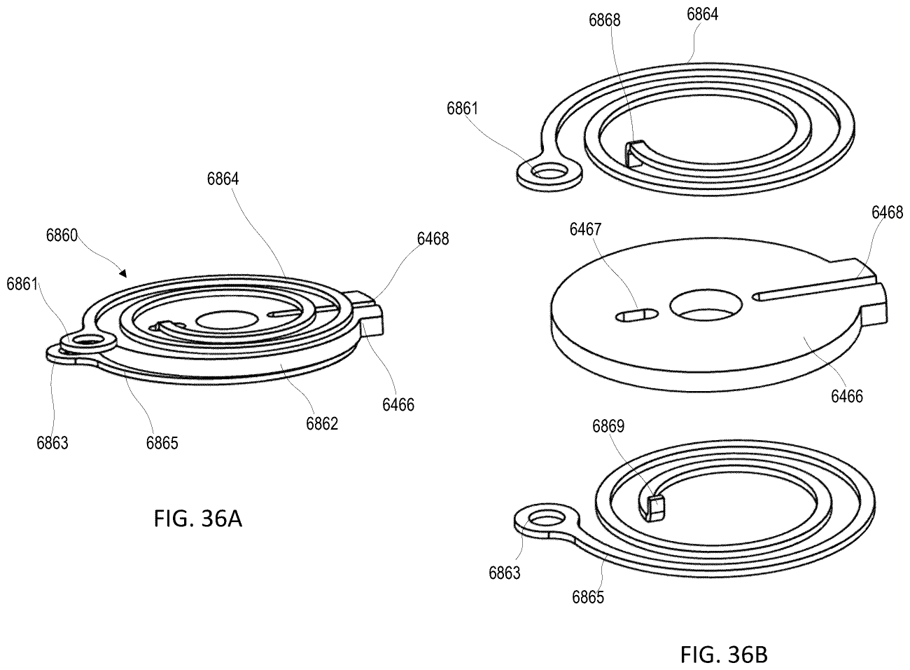

The intermediate coupling mechanism 6860 (also referred to as “torsion spring mechanism”) includes a first torsion spring 6864 and a second torsion spring 6865 coupled on opposite sides of the shape fiber carrier 6466. The torsion springs 6864, 6865 can be spiral torsion springs (also referred to as “clock springs”), helical torsion springs, torsional bars or fibers, or any other suitable torsion spring design. In this embodiment, the torsion spring mechanism includes two clock springs. The first clock spring 6864 includes a loop end 6861 and a tab portion 6868 at an opposite end of the loop end 6861 (see FIGS. 34 and 36B). Similarly, the second clock spring 6865 includes a loop end 6863 and a tab portion 6869 at an opposite end of the loop end 6863 (see FIGS. 34 and 36B). The loop end 6861 of the clock spring 6864 is coupled to a pin 6448 (see FIGS. 31-35) of the first tool member 6462 and the tab portion 6868 is engaged with the opening 6467 of the shape fiber carrier 6466. The loop end 6863 of the clock spring 6865 is coupled to a pin 6449 (see FIG. 35) of the second tool member 6482 and the tab portion 6869 is engaged with the opening 6467 of the shape fiber carrier 6466. The engagement of the tab portions 6868 and 6869 of the clock springs 6464 and 6465, respectively, within the opening 6467 of the shape fiber carrier 6466 acts upon the shape fiber carrier 6466 and causes it to rotate as the first tool member 6462 and/or second tool member 6482 are rotated.

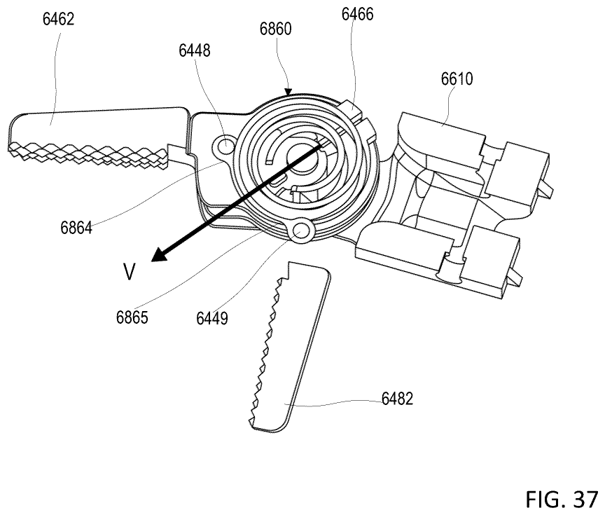

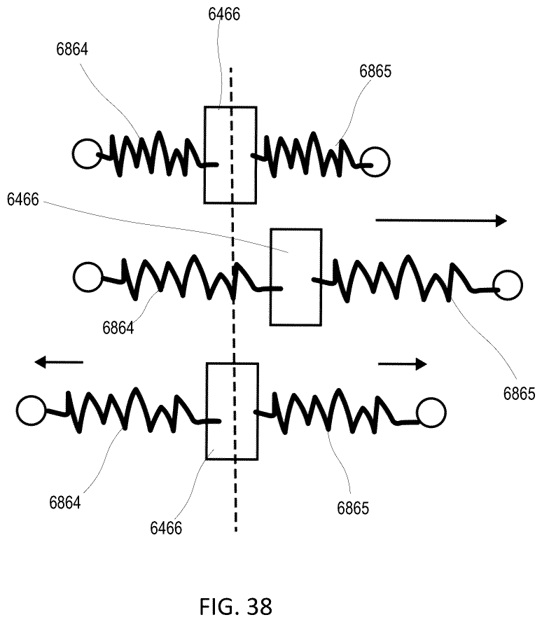

As shown schematically in FIG. 38, the opposing forces exerted by the clock springs 6864 and 6865 balance the position of the shape fiber carrier 6466 such that the shape fiber carrier 6466 (and corresponding shape fiber 6588) is aligned with a centerline between the jaws. Specifically, the opposing forces exerted by the clock springs 6864 and 6865 balance the shape fiber carrier 6466 such that a centerline CLsfc of the shape fiber carrier 6466 is maintained at approximately a center location between the first tool member 6462 and the second tool member 6482 as the first tool member 6462 and the second tool member 6482 move relative to each other or move relative to the distal link 6610. Similarly stated, the rotational position of the shape fiber carrier 6466 is associated with the average of the first yaw angle of the first tool member 6462 and the second yaw angle of the second tool member 6482. The orientation of the shape fiber carrier 6466 with respect to the average yaw angle is affected by the spring rate of the two clock springs 6864 and 6865. For example, if the clock springs 6864 and 6865 have the same spring rate and the friction and other losses are identical, then the opposing forces exerted on the shape fiber carrier 6466 will be identical and the shape fiber carrier 6466 will be maintained at the center location between the first tool member 6462 and the second tool member 6482. Slight differences in the spring rate between the clock springs (e.g., caused by manufacturing tolerances, etc.), however, can cause the rotational position of the shape fiber carrier 6466 to be slightly offset from the average of the first yaw angle of the first tool member 6462 and the second yaw angle of the second tool member 6482. Accordingly, in some embodiments, any of the instruments described herein (including the instrument 6400) can include an electronic circuit system as described herein (see e.g., the electronic circuit board 1815 and methods described below) that can contain a calibration module to ensure that an adjusted signal is produced that is representative of the average yaw angle. Thus, the shape fiber 6588 (coupled to the shape fiber carrier 6466) is oriented along the shape fiber vector V (shown in FIG. 37) associated with the average of the first yaw angle of the first tool member 6462 and the second yaw angle of the second tool member 6482, and outputs a signal associated with the vector V.

When one of the first tool member 6462 or the second tool member 6482 rotates relative to the other such that a direction of the average of the positions of the tool members 6462, 6482 changes (e.g., with respect to the distal wrist link 6610), the shape fiber carrier 6466 will move via the engagement between the torsion spring mechanism 6860 and the first tool member 6462 and the second tool member 6482. The shape fiber carrier 6466 rotates relative to the distal wrist link 6610 about the tool axis A1. This, in turn, causes the shape fiber 6588 to move. The rotation or orientation of the shape fiber carrier 6466 is associated with an average of a first yaw angle of the first tool member 6462 and a second yaw angle of the second tool member 6482. More specifically, the measurement of the position of the tool members 6462, 6482 is determined as the shape fiber 6588 bends in proportion to the average between the two tool members 6462, 6482. The use of the torsion spring mechanism 6860 is associated with the motion of first tool member 6462 and the second tool member 6482, and also the shape fiber 6588. In some embodiments, the clock spring design can also accommodate the limits on the bend radius of the shape fiber 6588 and to accommodate the straight section at the distal tip portion 6589 of the shape fiber 6588. When the shape fiber 6588 moves or bends, the shape fiber 6588 produces a signal related to the movement of the shape fiber 6588. In some embodiments, the signal produced by the shape fiber 6588 is associated with an average of a first yaw angle of the first tool member 6462 and a second yaw angle of the second tool member 6482.

As described herein, the torsion spring mechanism 6860 can allow for the use of a single shape fiber sensor (e.g., shape fiber 6588) to determine the position of the two tool members 6462 and 6482. The single shape fiber sensor can be used to determine position and orientation of the tool members 6462, 6482, with the shape fiber 6588 not being directly coupled to the tool members 6462, 6482. In addition to providing position and orientation information of the tool members 6462, 6482, in some embodiments, movement of the shape fiber carrier 6466 also causes the shape fiber to produce a signal related to movement of the distal wrist link 6610. In some embodiments, movement of the shape fiber carrier 6466 additionally causes the shape fiber 6588 to produce a signal related to movement of a proximal articulating joint and/or link such as the proximal wrist link and other flexible instrument portions (e.g., a flexible instrument shaft). Accordingly, the shape fiber 6588 may be used to determine position and orientation of the tool members 6462, 6482 as well as of other portions of the medical device.

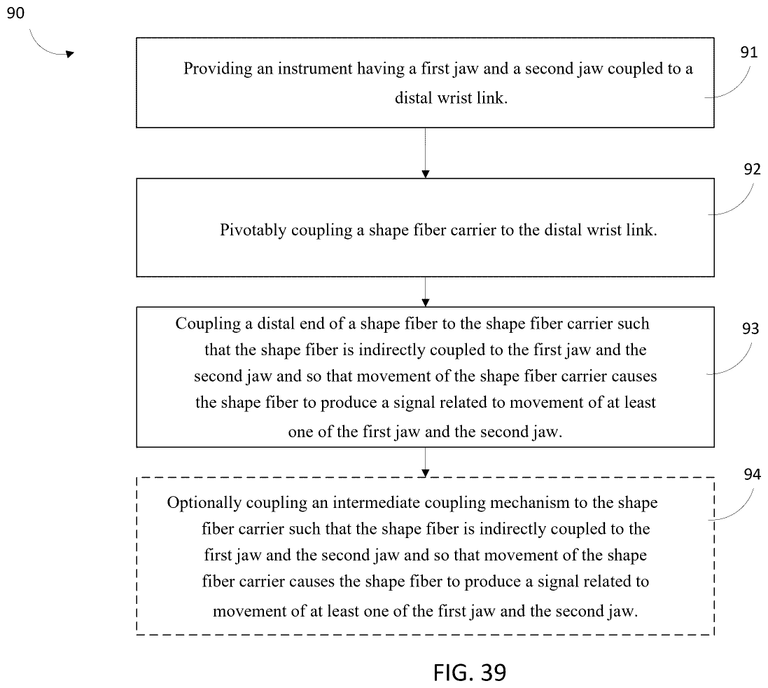

FIG. 39 is a flowchart illustrating a method of assembling a medical device as described herein having a shape fiber sensor (e.g., 2588, 3588, 4588, 5588, 6588) to produce a signal related to the movement of the shape fiber sensor that is associated with an average of a first yaw angle of a first tool member and a second yaw angle of a second tool member of the medical device. A method 90 includes at 91, providing an instrument having a first tool member (e.g., a first jaw) and a second tool member (e.g., a second jaw) coupled to a distal wrist link. At 92, a shape fiber carrier is pivotably coupled to the distal wrist link, wherein the shape fiber carrier defines a shape fiber lumen. At 93, a distal end of a shape fiber is coupled to the shape fiber carrier such that the shape fiber is indirectly coupled to the first jaw and the second jaw, and such that movement of the shape fiber carrier causes the shape fiber to produce a signal related to movement of at least one of the first jaw and the second jaw. For example, the distal end of the shape fiber can be fixedly coupled to the shape fiber carrier within the shape fiber lumen. At 94, an intermediate coupling mechanism is optionally coupled to the shape fiber carrier such that the shape fiber is indirectly coupled to the first jaw and the second jaw via the shape fiber carrier and the intermediate coupling mechanism. The intermediate coupling mechanism can include, for example, a differential with gear train, a cam mechanism, a torsion spring mechanism, or other suitable mechanism to function as described herein.

The medical devices described herein can be used to control the movement of the tool members (e.g., jaws) and processing of the signals generated by the shape fiber sensor (e.g., 2588, 3588, 4588, 5588, 6588). For example, a medical device can include a first tool member and a second tool member each coupled to a distal wrist link, a shape fiber carrier pivotably coupled to the distal wrist link, and a shape fiber fixedly coupled within a shape fiber lumen of the shape fiber carrier, wherein the shape fiber is indirectly coupled to the first tool member and the second tool member (e.g., the shape fiber is spaced apart from the tool members). A method of using the medical device can include rotating at least one of the first tool member or the second tool member such that the shape fiber carrier is rotated and the shape fiber produces a signal proportional to the movement of the at least one of the first tool member or the second tool member. For example, the signal produced by the shape fiber can include a signal associated with an average of a first yaw angle of the first jaw and a second yaw angle of the second jaw.

In some embodiments, any of the instruments or systems described herein can include an electronic circuit system (e.g., an instrument circuit board or a controller) to produce an output from the shape fiber. The output can be used, for example to produce a haptic feedback at the user control unit 1100. In some embodiments, any of the instruments can include a circuit board that includes a memory device and/or a process that can be calibrated to during manufacturing so that that the output from the shape fiber sensor is associated with an average of an average of a first yaw angle of the first jaw and a second yaw angle of the second jaw. For example, FIG. 40 is a schematic diagram of one embodiment of suitable components that may be included within an electronic circuit board 1815 is illustrated. In some embodiments, the electronic circuit board 1815 is positioned within the instrument 1400 (e.g., within a proximal mechanical structure). In other embodiments, the electronic circuit board 1815 is a component of other portions of the surgical system 1000, such as the user control unit 1100 and/or the optional auxiliary equipment unit 1150. However, the electronic circuit board 1815 may also include distributed computing systems wherein at least one aspect of the electronic circuit board 1815 is at a location which differs from the remaining components of the surgical system 1000 for example, at least a portion of the electronic circuit board 1815 may be an online controller.

As depicted, the electronic circuit board 1815 includes one or more processor(s) 1802 and associated memory device(s) 1804 configured to perform a variety of computer implemented functions (e.g., performing the methods, steps, calculations and the like and storing relevant data as disclosed herein). Additionally, in some embodiments, the electronic circuit board 1815 includes a jaw sensor module 1806 to facilitate communications between the electronic circuit board 1815 and the various components of the surgical system 1000, such as the shape fiber sensors described herein.

As used herein, the term “processor” refers not only to integrated circuits referred to in the art as being included in a computer, but also refers to a controller, a microcontroller, a microcomputer, a programmable logic controller (PLC), an application specific integrated circuit, and other programmable circuits. Additionally, the memory device(s) 1804 may generally comprise memory element(s) including, but not limited to, computer readable medium (e.g., random access memory (RAM)), computer readable nonvolatile medium (e.g., a flash memory), a floppy disc, a compact disc read only memory (CD ROM), a magneto optical disc (MOD), a digital versatile disc (DVD) and/or other suitable memory elements. Such memory device(s) 1804 may generally be configured to store suitable computer readable instructions that, when implemented by the processor(s) 1802, configure the controller 1800 to perform various functions.

In some embodiments, the electronic circuit board 1815 can be operatively coupled to (or include) a haptic feedback controller 1820. The haptic feedback controller 1820 may be configured to deliver a haptic feedback to the operator based on inputs received from any of the shape fiber sensors described herein, which are included in the instrument 1400.

The jaw sensor module 1806 may include a sensor interface 1810 (e.g., one or more analog to digital converters) to permit signals transmitted from one or more sensors (e.g., shape fiber sensors 2588, 3588, 4588, 5588, 6588) to be converted into signals that can be understood and processed by the processors 1802. The sensors may be communicatively coupled to the communication module 1806 using any suitable means. For example, the sensors may be coupled to the communication module 1806 via a wired connection and/or via a wireless connection, such as by using any suitable wireless communications protocol known in the art. The jaw sensor module 1806 may also include an output module 1812 configured to generate outputs associated with the shape fiber sensors. For example, the outputs generated can be one or more signals associated with an average yaw position of the jaws.

Additionally, in some embodiments, the communication module 1806 includes a calibration module 1814 configured to adjust any of the signals of the shape fiber sensors produced herein. For example, in some embodiments, the calibration module 1814 can include a calibration table or a calibration curve (e.g., an equation) that relates the output from a shape fiber (e.g., the shape fiber sensors 2588, 3588, 4588, 5588, 6588) with the actual average of the first yaw angle of the first tool member and the second yaw angle of the second tool member as measured during assembly of the instrument. Similarly stated, the calibration table (or calibration curve) can provide a correlation between the orientation of the shape fiber sensor (e.g., the orientation of the housing 4280, or the shape fiber carrier 6466) and the actual average of the first yaw angle of the first tool member and the second yaw angle of the second tool member. In some embodiments, the calibration table (or calibration curve) can provide an amplification factor to account for the reduced range of motion of the shape fiber sensor as compared to the range of motion of the jaws. For example, in some embodiments where the shape fiber sensor moves at less than a one-to-one relationship with the movement of the jaws (e.g., embodiments that employ a differential), the calibration table (or calibration curve) can include a multiplication factor. In use, the calibration module can facilitate methods of producing an adjusted output signal that is specific to (or unique to) the instrument. In this manner, the calibration module and methods can ensure that the output of the shape fiber sensor accounts for slight differences (e.g., due to part-to-part variability, slight differences in friction, manufacturing tolerances, etc.) in the components of any of the intermediate coupling mechanisms described herein. The calibration module and methods can also account for differences in the rotational range of movement between the jaws and the shape fiber sensor (e.g., in embodiments where a differential is employed).

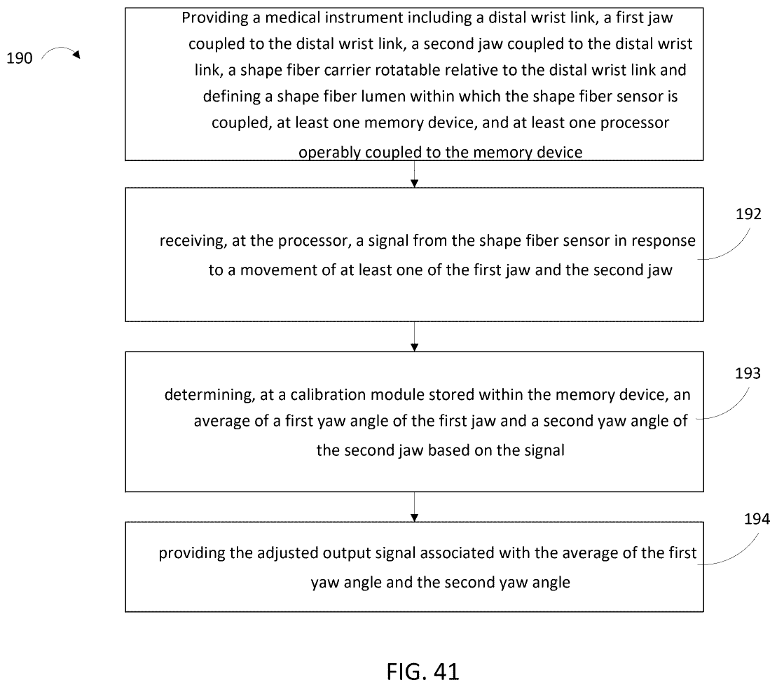

In some embodiments, any of the instruments described herein can include an electronic circuit system (e.g., the system 1815) and can perform methods of adjusting a signal output from a shape fiber. For example, FIG. 41 is a flow chart of a method 190 of producing an adjusted output signal from a shape fiber sensor of a medical instrument, according to an embodiment. The medical instrument can be any of the medical instruments described herein and includes a distal wrist link, a first jaw coupled to the distal wrist link, a second jaw coupled to the distal wrist link, a shape fiber carrier rotatable relative to the distal wrist link and defining a shape fiber lumen within which the shape fiber sensor is coupled, at least one memory device, and at least one processor operably coupled to the memory device. The method includes receiving, at the processor, a signal from the shape fiber sensor in response to a movement of at least one of the first jaw and the second jaw, at 192. The method includes determining, at a calibration module stored within the memory device, an average of a first yaw angle of the first jaw and a second yaw angle of the second jaw based on the signal, at 193. The method includes providing the adjusted output signal associated with the average of the first yaw angle and the second yaw angle, at 194.

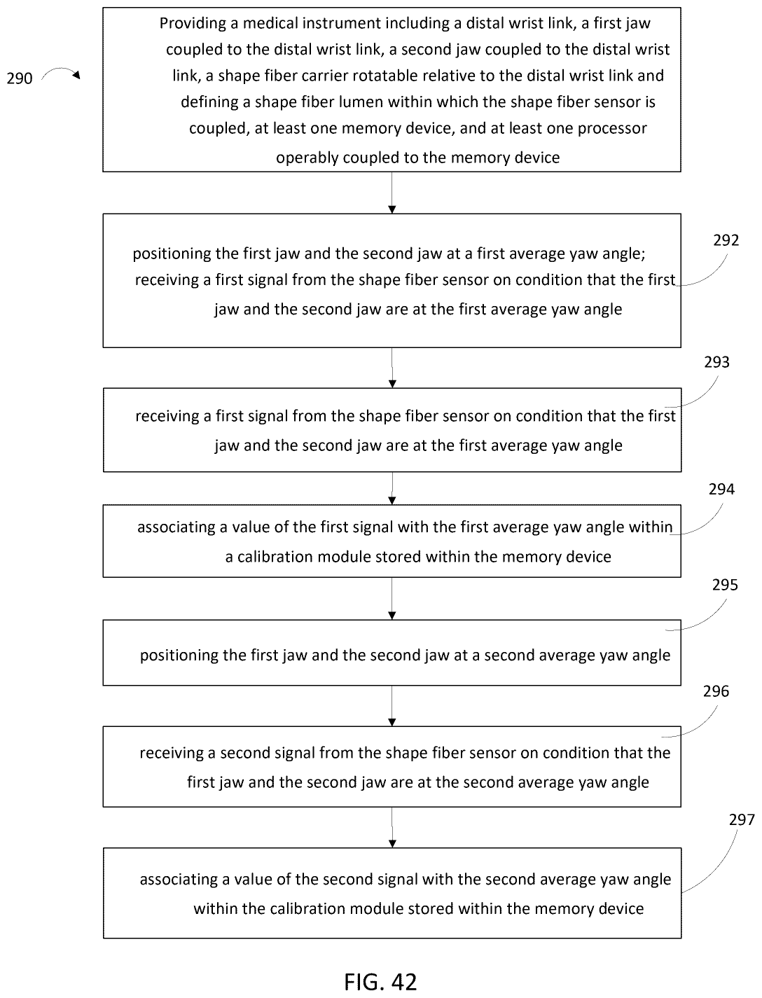

FIG. 42 is a flow chart of a method 290 of calibrating a shape fiber sensor of a medical instrument, according to an embodiment. The medical instrument can be any of the medical instruments described herein and includes a distal wrist link, a first jaw coupled to the distal wrist link, a second jaw coupled to the distal wrist link, a shape fiber carrier rotatable relative to the distal wrist link and defining a shape fiber lumen within which the shape fiber sensor is coupled, at least one memory device, and at least one processor operably coupled to the memory device. The method includes positioning the first jaw and the second jaw at a first average yaw angle, at 292. A first signal is received from the shape fiber sensor on condition that the first jaw and the second jaw are at the first average yaw angle, at 293. The method includes associating a value of the first signal with the first average yaw angle within a calibration module stored within the memory device, at 294. The first jaw and the second jaw are positioned at a second average yaw angle, at 295. The method includes receiving a second signal from the shape fiber sensor on condition that the first jaw and the second jaw are at the second average yaw angle, at 296. The method includes associating a value of the second signal with the second average yaw angle within the calibration module stored within the memory device, at 297.

While various embodiments have been described above, it should be understood that the embodiments have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument described herein can be constructed from any material, such as medical grade stainless steel, nickel alloys, titanium alloys or the like. Further, any of the links, tool members, tension elements, or components described herein can be constructed from multiple pieces that are later joined together. For example, in some embodiments, a link can be constructed by joining together separately constructed components. In other embodiments however, any of the links, tool members, tension elements, or components described herein can be monolithically constructed.

Although the instruments are generally shown as having an axis of rotation of the tool members (e.g., axis A1) that is normal to an axis of rotation of the wrist members (e.g., axis A2, axis A3), in other embodiments any of the instruments described herein can include a tool member axis of rotation that is offset from the axis of rotation of the wrist assembly by any suitable angle.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A medical device, comprising:
a distal wrist link;
a first jaw and a second jaw each coupled to the distal wrist link;
a shape fiber carrier pivotably coupled to the distal wrist link and defining a shape fiber lumen; and
a shape fiber having a distal end fixedly coupled within the shape fiber lumen;
the shape fiber being indirectly coupled to the first jaw and the second jaw,
wherein movement of the shape fiber carrier causes the shape fiber to produce a signal related to movement of at least one of the first jaw or the second jaw.

2. The medical device of claim 1, further comprising:
an intermediate coupling mechanism coupled to the shape fiber carrier; and
the shape fiber being indirectly coupled to the first jaw and the second jaw via the shape fiber carrier and the intermediate coupling mechanism.

3. The medical device of claim 2, wherein:
the intermediate coupling mechanism is a differential coupled between the at least one of the first jaw or the second jaw and the shape fiber carrier; and
the shape fiber is indirectly coupled to the first jaw and the second jaw via the differential and the shape fiber carrier.

4. The medical device of claim 2, wherein:
the intermediate coupling mechanism comprises a cam mechanism indirectly coupling the shape fiber to the at least one of the first jaw or the second jaw.

5. The medical device of claim 2, wherein:
the intermediate coupling mechanism comprises a torsion spring indirectly coupling the shape fiber to the at least one of the first jaw or the second jaw.

6. The medical device of claim 1, wherein:
the signal produced by the shape fiber includes a signal associated with an average of a first yaw angle of the first jaw and a second yaw angle of the second jaw.

7. The medical device of claim 1, wherein:
the shape fiber carrier is pivotably coupled to the distal wrist link about a first axis; and
the first jaw and the second jaw are each pivotably coupled to the distal wrist link about a second axis, the second axis offset from the first axis.

8-9. (canceled)

10. The medical device of claim 3, wherein:
the differential comprises a housing, a first planet gear, a second planet gear, and a ring gear;
the medical device further comprises: a gear train, a first sun gear and a second sun gear;
the first sun gear is integral to the first jaw and is coupled to the housing of the differential, the second sun gear is integral to the second jaw and is coupled to the housing of the differential;
the ring gear is integral to the housing and coupled to the gear train, and the gear train is coupled to the shape fiber carrier;
the housing of the differential is configured to rotate when one of the first jaw or the second jaw rotates relative to the other, rotation of the housing causes rotation of the shape fiber carrier and the shape fiber; and
an orientation of the housing is associated with an average of a first yaw angle of the first jaw and a second yaw angle of the second jaw.

11. The medical device of claim 10, wherein the shape fiber produces a signal associated with an average of a yaw angle of the first jaw and a yaw angle of the second jaw based on the orientation of the housing.

12. The medical device of claim 3, wherein:
the movement of the shape fiber carrier is limited by the differential.

13. (canceled)

14. The medical device of claim 3, wherein:
the shape fiber carrier includes a gear operatively coupled to the differential.

15. The medical device of claim 1, wherein:
the movement of the shape fiber carrier causes the shape fiber to produce a signal related to movement of the distal wrist link.

16. The medical device of claim 1, wherein:
the movement of the shape fiber carrier causes the shape fiber to produce a signal related to movement of a proximal articulating joint.

17. A medical device, comprising:
a distal wrist link;
a first jaw and a second jaw each coupled to the distal wrist link;
a position sensor coupled to the distal wrist link; and
a differential coupled to the position sensor, the first jaw, and the second jaw;
wherein movement of one of the first jaw or the second jaw relative to the other produces a sensor movement of the position sensor via the differential, and the position sensor produces a signal corresponding to the sensor movement.

18. The medical device of claim 17, wherein:
the signal produced by the position sensor is associated with an average of a first yaw angle of the first jaw and a second yaw angle of the second jaw.

19. The medical device of claim 17, wherein:
the position sensor is a shape fiber, the medical device includes a shape fiber carrier coupled to the differential, and the shape fiber is at least partially disposed within a lumen of the shape fiber carrier.

20. The medical device of claim 19, wherein:
the differential includes a housing, a first planet gear, a second planet gear, and a ring gear;
the medical device further comprises a gear train, a first sun gear and a second sun gear;
the first sun gear is integral to the first jaw and coupled to the housing of the differential, the second sun gear is integral to the second jaw and coupled to the housing of the differential;
the ring gear is coupled to the housing and coupled to the gear train, and the gear train is coupled to the shape fiber carrier;
the housing of the differential is configured to rotate when one of the first jaw or the second jaw rotates relative to the other, rotation of the housing causes rotation of the position sensor; and
an orientation of the housing is associated with an average of a first yaw angle of the first jaw and a second yaw angle of the second jaw.

21. The medical device of claim 20, wherein the position sensor produces a signal associated with the average of the first yaw angle of the first jaw and the second yaw angle of the second jaw based on the orientation of the housing.

22. (canceled)

23. The medical device of claim 19, wherein:
the movement of the shape fiber carrier is limited by the differential.

24. The medical device of claim 23, wherein:
the movement of the shape fiber carrier is limited to +/−45 degrees relative to a centerline of the shape fiber carrier.

25. The medical device of claim 17, wherein:
the position sensor is indirectly coupled the first jaw and the second jaw such that an angular position of the position sensor is associated with an average of a first yaw angle of the first jaw and a second yaw angle of the second jaw.

26-37. (canceled)
